(19) United States
(12) Patent Application Publication
(10) Pub. No.: US 2013/0172690 A1
(43) Pub. Date: Jul. 4, 2013

(54) APPARATUS, SYSTEM AND METHOD FOR DETECTION AND DELIVERY OF A MEDICINAL DOSE

(71) Applicants: Lawrence Arne, Palo Alto, CA (US); Benedict Costello, Berkeley, CA (US); Yuriy Mestman, Mountain View, CA (US); Timothy Robertson, Belmont, CA (US); George Savage, Portola Valley, CA (US); Angela Strand, San Francisco, CA (US); Todd Thompson, San Jose, CA (US); Mark Zdeblick, Portola Valley, CA (US); William McAllister, Saratoga, CA (US)

(72) Inventors: Lawrence Arne, Palo Alto, CA (US); Benedict Costello, Berkeley, CA (US); Yuriy Mestman, Mountain View, CA (US); Timothy Robertson, Belmont, CA (US); George Savage, Portola Valley, CA (US); Angela Strand, San Francisco, CA (US); Todd Thompson, San Jose, CA (US); Mark Zdeblick, Portola Valley, CA (US); William McAllister, Saratoga, CA (US)

(73) Assignee: PROTEUS DIGITAL HEALTH, INC., Redwood City, CA (US)

(21) Appl. No.: 13/640,699

(22) PCT Filed: Apr. 11, 2011
(86) PCT No.: PCT/US2011/031986

§ 371 Date: Feb. 19, 2013

Related U.S. Application Data
(60) Provisional application No. 61/322,893, filed on Apr. 11, 2010.
(60) Provisional application No. 61/357,506, filed on Jun. 22, 2010.
(60) Provisional application No. 61/373,803, filed on Aug. 13, 2010.
(60) Provisional application No. 61/377,072, filed on Aug. 25, 2010.

(51) Int. Cl. A61B 5/00; A61B 5/053

(52) U.S. Cl. CPC A61B 5/4833; A61B 5/053
USPC 600/301; 600/547

(57) ABSTRACT

An apparatus is disclosed as part of a system for tracking and confirming delivery of a medicinal dose to a user. The apparatus includes a detector. The detector is secured to and communicatively coupled to the user and is capable of detecting a current flow through the user's body. The current flow is produced when the user makes contact with the apparatus. The apparatus includes at least two contact areas connected to a power source where a circuit and, hence, a current path is completed through the user's body as the user makes contact with the apparatus. The current flow is detected by the detector, which is coupled to the user. Also disclosed is an apparatus for tracking and confirming delivery of a medicinal dose to a user where the apparatus includes an acoustic detector. Upon loading the dose into a chamber an acoustic vibration is generated. The vibration is detected and correlated with a current flow that is produced when the user makes contact with the apparatus. The combined event of vibration detection and current flow detection confirms that the dose is loaded and the user is in contact with the apparatus and ready to receive the dose.

### CROSS REFERENCE TO RELATED APPLICATIONS

This application claims the benefit of U.S. Provisional Patent Application Ser. No. 61/322,893, filed Apr. 11, 2010 and entitled “System and Method for Delivery of an Inhalable Dose”; U.S. Provisional Patent Application Ser. No. 61/357,506, filed Jun. 22, 2010 and entitled “System and Method for Detection of Delivery of an Inhalable Dose”; and U.S. Provisional Patent Application Ser. No. 61/373,803, filed Aug. 13, 2010 and entitled “System and Method for Delivery and Detection of an Inhalable Dose”; and U.S. Provisional Patent Application Ser. No. 61/377,072, filed Aug. 25, 2010 and entitled “System and Method for Patient Access Port Reporting” each of which is hereby incorporated by reference in its entirety.

### FIELD OF THE INVENTION

The present invention is related to electronic systems and, more specifically, to electronic systems for determining and confirming delivery of a dose of medication.

### INTRODUCTION

Commonly used medication delivery systems include a chamber for storing the dose. As needed, a patient will use the system to take a dose of the medication. However, there is no accurate way of determining if the patient took the dose or when the patient took the dose. For example, there are delivery systems that track the number of times a dose was dispensed or delivered. However, the delivery systems lack the ability to determine if and when the doses were delivered to a patient. For example, a patient may discharge the delivery system several times in a short period of time to give the appearance that several doses were taken over a period of time. There are some delivery systems that time-stamp the delivery, but lack the ability to determine if the patient actually received the dose as required. For example, the patient may discharge the delivery system over a long period of time without actually taking or inhaling the dose to give the appearance that the doses were taken regularly over a longer period of time. Thus, these delivery systems are not capable of determining if the patient actually received the dose. Furthermore, there is no accurate way of determining if the dose that was delivered was actually delivered to the patient as intended. For example, the delivery system may have been discharged by someone other than the patient. Additionally, in some instances, the delivery systems may have been accidentally discharged.

Therefore, what is needed is a system and method for determining if a medicinal dose was delivered to a patient as intended as well as tracking the delivery time and thereby providing an accurate determination of when the patient actually received the dose.

### SUMMARY

In accordance with aspects of the systems and teaching of the present invention, a system and method are provided that track the delivery time of a medicinal dose to the patient as well as providing confirmation that the patient actually received the dose. The system includes a detector and an apparatus. The detector is communicatively coupled to the user and is capable of detecting a current flow through the user's body. The current flow is produced when the user makes contact with the apparatus. The apparatus includes at least two contact areas connected to a power source such that a circuit and, hence, a current path is completed through the user's body as the user makes contact with each of the two contact areas and a current flow is detected by the detector, which is coupled to the user.

In another aspect, the system includes a detector and an apparatus with an acoustic unit. The detector is communicatively coupled to the user and is capable of detecting a current flow through the user's body as well as acoustic information produced by the acoustic unit. The current flow is produced when the user makes contact with the apparatus. The apparatus includes at least two contact areas connected to a power source such that a circuit and, hence, a current path is completed through the user's body as the user makes contact with each of the two contact areas and a current flow is detected by the detector, which is coupled to the user.

Notwithstanding the claims, the present invention is also defined by the following clauses:
1. An apparatus for delivering a dose to a user and track the timing of the delivery of the dose, the apparatus comprising:

a detector configured to couple to the user;

an apparatus, wherein the apparatus comprises:
a housing defining a chamber to store the dose;
at least two contact areas positioned on the housing wherein the contact areas are near or on the exterior of the housing and the at least two contact areas are electrically isolated from each other;
a power source secured to the housing and including a positive phase terminal and a negative phase terminal, wherein at least one contact area is electrically coupled to the positive phase terminal and at least one other contact area is electrically coupled to the negative phase terminal; and
wherein a current path is completed through the user's body as the user makes contact with each of the two contact areas and current flow is detected by the detector that is coupled to the user.
   2. The apparatus of clause 1, wherein the dose is an inhalable dose or wherein the dose is an ingestible dose.
   4. The apparatus of clause 1 or 2, further comprising a control module electrically connected between the power source and one of the at least two contact areas, wherein the control module is configured to control information associated with the apparatus, preferably wherein the control module is electrically coupled to the at least two contact areas and to both terminals of the power source, for example wherein the control module is configured to vary the conductance of the current path to encode information in the current flow, preferably wherein the apparatus further comprises a transceiver electrically coupled to the control module wherein information can be transmitted and/or received from the apparatus to the detector other than through the current flow.
   5. The apparatus of clause 4, wherein the detector comprises:

a hermetically sealed housing;

a power source secured within the housing;

a processor electrically coupled to the power source and secured within the housing;

at least one sensing probe secured to the housing wherein the probe is electrically coupled to the processor so that the processor detects physiological parameters associated with the user and the current flow through the user; and

a memory unit electrically coupled to the processor and secured to or within the housing to store data;

wherein the transceiver is electrically coupled to the processor and secured within the housing to receive and decode information transmitted from the apparatus.

6. The apparatus of clause 4 or 5 wherein the detector is configured to be implanted within the user's body or on the user's skin, wherein the probe is at least partially exposed to contact the user's tissue or skin.
7. The apparatus of any of the clauses 4-6 wherein the transceiver is communicatively coupled to a data management center to provide two-way wireless communication of the data from the detector to the data management center.
8. The apparatus according to any of the preceding clauses further comprising an acoustic unit secured to the housing, preferably wherein the acoustic unit comprises:

a support layer comprising an adhesive layer on one surface for securing the acoustic unit to the apparatus;

a vibration detection unit secured to the support layer to detect acoustic information produced by the apparatus and the user inhaling through the apparatus and produce a detection signal;

a controller secured to the support layer and in communication with the vibration detection unit, wherein the controller receives the detected signal from the vibration detection unit and produces a digital signal representing the detected signal;

a sound generation unit secured to the support layer and in communication with the controller, wherein the sound generation unit receives the digital signal and produces the acoustic signal; and

a top layer secured to the support layer to define a cavity that contains and protects the vibration unit, the controller, and the sound generation unit within the cavity,

wherein the acoustic signal represents information associated with the inhalable dose being loaded into the chamber and the user inhaling through the apparatus and wherein the acoustic signal is detected by the detector to confirm delivery of the dose.

9. The apparatus of clause 8, wherein the acoustic unit is coupled to a control module secured to the housing.
10. The apparatus of clause 8 or 9, wherein the acoustic unit provides an activation signal to the control module upon detection of a vibration representing the loading of a medication into the chamber.
11. The apparatus of any of the clauses 8-11 wherein a sound of the dose being loaded into the chamber activates the control module and wherein a current path is completed through the user's body as the user makes contact with each of the contact areas indicating that the user is about the receive a dose of a medication.
12. The apparatus of any of the clauses 8-12 wherein the acoustic unit provides an activation signal to the control module and the control module records acoustic information associated with the user inhaling, wherein the information is provided to the control module through the acoustic detector.
13. The apparatus according to any of the preceding clauses 8-12 wherein the control module provides a unique time stamp associated with the delivery of the dose and wherein the control module receives an identifier signal that is associated with the user wherein the combination of the time stamp and the identifier signal confirms deliver of the dose to the user.
14. The apparatus according to any of the preceding clauses 4-13 wherein the transceiver is electrically coupled to the control module and secured to the housing and wherein the transceiver allows the apparatus to transmit and/or receive information associated with the delivery of the dose to the user.
15. The apparatus of clause 14, wherein the transceiver comprises at least one of an optical transmitter module for optical communication and a wireless transmitter module for wireless communication.
16. The apparatus of clauses 14 or 15, wherein the transceiver encodes information from the memory unit and transmits that information to a system external to the apparatus.
17. The apparatus of clause 16, wherein the transceiver sends an activation signal to the external system once a circuit is completed through the user's body wherein the activation signal is an indicator that the user is prepared to initiate delivery of the dose.
18. The apparatus according to any of the preceding clauses 8-17 wherein the acoustic unit is activated by a processor to detect acoustic information from the user's lungs upon the acoustic unit receiving the activation signal from the apparatus.
19. A system to deliver a dose to a user and to confirm delivery of the dose, the system comprising an apparatus preferably according to any of the preceding clauses and a detector configured to couple to the user.
20. A system according to clause 21 wherein the detector includes a capacitive coupler.
21. A system according to clauses 19 or 20 further comprising,

a housing defining a chamber to store the dose;

a power source secured to the housing and including a positive terminal and a negative terminal, wherein the power source comprises an isolating source that produces a carrier wave;

a control module electrically coupled to the power source, wherein the control module alters the characteristics of the isolating source to encode information in the carrier wave; and

at least two areas positioned on the housing wherein one area is a partially exposed contact area and one area is capacitive coupled area, wherein the two areas are electrically isolated from each other,

wherein one output of the isolating source is coupled to the contact area and the other output of the isolating source is coupled to the capacitive coupled area,

wherein the contact area is touched by the user and the capacitive coupled area is capacitively coupled to the capacitive coupler worn by the user, and

wherein a portion of the carrier wave's path is through the user's body using the contact area and a portion of carrier wave's path is through capacitive conductance using the capacitive coupling between the capacitive coupled area and the capacitive coupler worn by the user.

22. The system of clause 21, wherein the housing defines an aperture to generate an acoustic wave as the user inhales through the apparatus and wherein the detector further comprises an acoustic unit for detecting acoustic wave associated with the user inhaling through the apparatus, which acoustic waves traveling through the user's body and through the air, wherein the acoustic unit correlates the acoustic wave through the user's body and the acoustic wave through the air to confirm delivery of the dose to the user.
23. The system according to any of the clauses 19-22 further comprising a vibration detection unit to detect acoustic information and produce a detection signal;

a controller in communication with the vibration detection unit, wherein the controller receives the detection signal from the vibration detection unit and produces a digital signal representing the detection signal;

a sound generation unit secured to the support layer and in communication with the controller, wherein the sound generation unit receives the digital signal and produces an acoustic signal that indicates delivery of the dose.

24. An apparatus for detection of delivery of a dose to a user, the apparatus comprising:

a vibration detection unit to detect acoustic information and produce a detection signal;

a controller in communication with the vibration detection unit, wherein the controller receives the detection signal from the vibration detection unit and produces a digital signal representing the detection signal;

a sound generation unit secured to the support layer and in communication with the controller, wherein the sound generation unit receives the digital signal and produces an acoustic signal that indicates delivery of the dose.

25. The apparatus of clause 24, further comprising a transceiver in communication with the controller, wherein the transceiver receives the digital signal and communicates with a wireless apparatus to indicate the inhalation event has occurred.
26. The apparatus of clause 24 or 25, further comprising a transconduction unit in communication with the controller, the transconduction unit comprising:

at least two contact areas electrically isolated from each other and positioned to allow contact with each of the contact areas;

a power source, wherein at least one contact area is electrically coupled to one terminal of the power source and at least one other contact area is electrically coupled to another terminal of the power source,

wherein a current path is completed through the user's body to allow current flow as the user makes contact with each of the two contact areas and current flow is detected by the controller.

27. The apparatus of clause 26, wherein the transconduction unit further comprises a control module electrically connected between the power source and one of the at least two contact areas, wherein the control module is configured to encode information in the current flow.
28. A method for recording the time that a dose is taken by a user, the method comprising the steps of:

activating a power module of an apparatus when the user makes contact with the exterior of the apparatus in such a manner to complete a circuit to allow for current flow between two terminals of the power module and through the user's body;

altering the current characteristics through changes in a conductance of the circuit that is formed through the user's body using a conductance control module;

detecting the current characteristics through the user's body using a detector, wherein the current characteristics comprises information associated with at least one of the apparatus and the dose; and

recording the timing of delivery of the inhalable dose.

29. The method of clause 28, comprising:

generating an acoustic signal using an acoustic unit; and

detecting the acoustic signal using an acoustic unit, wherein the acoustic signal comprises information associated with delivery of the dose to the user.

30. Use of a system or apparatus according to any of the preceding clauses for delivering a dose to a user and tracking the timing of the delivery of the dose, or for delivering a dose to a user and confirming delivery of the dose.

### BRIEF DESCRIPTION OF THE FIGURES

The novel features of the various aspects of the present invention are set forth with particularity in the appended claims. The various aspects, however, both as to organization and methods of operation, are described herein by way of example in conjunction with the following figures and corresponding description, where like reference numbers refer to like elements throughout.

FIG. 1A is an illustrative example of a user wearing a detector and making contact with an apparatus, which is shown in block diagram form, which delivers an inhalable dose in accordance with the teachings of the present invention.

FIG. 1B shows one possible physical shape for the apparatus of FIG. 1A, in accordance with the teachings of the present invention.

FIG. 1C shows one possible physical shape for the apparatus of FIG. 1A, in accordance with the teachings of the present invention.

FIG. 1D shows one possible physical shape for the apparatus of FIG. 1A, in accordance with the teachings of the present invention.

FIG. 2A is an alternative aspect of the apparatus of FIG. 1A shown in block diagram form and in accordance with the teachings of the present invention.

FIG. 2B is an alternative aspect of the apparatus of FIG. 1A shown in block diagram form and in accordance with the teachings of the present invention.

FIG. 3A is an illustrative example of a user wearing a detector and making contact with an apparatus, which is shown in block diagram form, which delivers an inhalable dose in accordance with the teachings of the present invention.

FIG. 3B shows another possible physical shape for the apparatus of FIG. 3A comprising an acoustic unit, in accordance with the teachings of the present invention.

FIG. 3C shows another possible physical shape for the apparatus of FIG. 3A comprising an acoustic unit, in accordance with the teachings of the present invention.

FIG. 3D shows another possible physical shape for the apparatus of FIG. 3A comprising an acoustic unit, in accordance with the teachings of the present invention.

FIG. 3E is an illustrative example of a user wearing a detector and making contact with an apparatus, which is shown in block diagram form, which delivers an inhalable dose in accordance another aspect of the present invention.

FIG. 3F is a side view illustration of a portion of the apparatus of FIG. 3E.

FIG. 4A is an alternative aspect of the apparatus of FIG. 3A comprising an acoustic unit shown in block diagram form and in accordance with the teachings of the present invention.

FIG. 4B is an alternative aspect of the apparatus of FIG. 3A comprising an acoustic unit shown in block diagram form and in accordance with the teachings of the present invention.

FIG. 4C is a top view illustration of an acoustic unit that in accordance with one aspect of the present invention.

FIG. 4D is an illustration of an acoustic unit in accordance with one aspect of the present invention.

FIG. 4E is an illustration of an acoustic unit in accordance with one aspect of the present invention.

FIG. 5A is an illustrative example of a user wearing a detector and making contact with an apparatus, which is shown in block diagram form in accordance with the teachings of the present invention.

FIG. 5B shows one possible physical shape for the apparatus of FIG. 5A with an apparatus electrically connected to contact areas in accordance with the teachings of the present invention.

FIG. 5C shows one possible physical shape for the apparatus of FIG. 5A with an apparatus electrically coupled to contact areas in accordance with the teachings of the present invention.

FIG. 5D shows one possible physical shape for the apparatus of FIG. 5A, with an apparatus and an acoustic unit, each electrically coupled to contact areas in accordance with the teachings of the present invention.

FIG. 5E shows an acoustic unit in accordance with one aspect and teaching of the present invention.

FIG. 5F shows an acoustic unit in accordance with another aspect and teaching of the present invention.

FIG. 6A is the apparatus of FIGS. 5B, 5C, and 5D in block diagram form and in accordance with one aspect of the teachings of the present invention.

FIG. 6B is the apparatus of FIGS. 5B, 5C, and 5D in block diagram form and in accordance with another aspect of the teachings of the present invention.

FIG. 7A is an illustration of the opening of the apparatus of FIGS. 1A, 3A, 5A in accordance with another aspect of the present invention, wherein a diaphragm is positioned near the opening of the apparatus through which the inhalable dose is dispensed.

FIG. 7B is an illustration of the diaphragm of the apparatus of FIG. 7A, wherein the diaphragm portions are parted as the dose is inhaled by the user such that a beam of light is interrupted and the interruption is detected.

FIG. 7C is an illustration of the diaphragm of the apparatus of FIG. 7A being parted as the dose is inhaled by the user such that a flexing motion and parting of the diaphragm portions cause an interruption in a connection.

FIG. 8 is a block diagram illustration of any one of the detectors of FIGS. 1A, 3A, 5A.

FIG. 9 is a block diagram illustration of a processor of FIG. 8.

FIG. 10 shows an example of a user wearing a detector and making contact with an apparatus as the user inhales to receive an inhalable dose in accordance with the teachings of the present invention.

FIG. 11 shows an access apparatus with the ability to record the timing of certain events associated with maintaining the access apparatus.

FIG. 12A is an illustrative example of a user wearing a detector and making contact with an apparatus, which is shown in block diagram form, which delivers a medicinal dose in accordance with the teachings of the present invention.

FIG. 12B shows one aspect of the medication delivery apparatus of FIG. 12A for delivering an individual pill from a reservoir of pills, in accordance with the teachings of the present invention.

FIG. 12C illustrates one aspect of the medication delivery apparatus of FIG. 12A for delivering an individual pill from a reservoir of pills, in accordance with the teachings of the present invention.

FIG. 12D illustrates one aspect of the medication delivery apparatus of FIG. 12A for delivering an individual pill from a reservoir of individually sealed pills, in accordance with the teachings of the present invention.

FIG. 13A is an illustrative example of a user wearing a detector and making contact with an apparatus, which is shown in block diagram form, which delivers a medicinal dose in accordance with the teachings of the present invention.

FIG. 13B illustrates one aspect of the medication delivery apparatus of FIG. 13A for delivering an individual pill from a tape comprising a plurality of pills sealed therein, in accordance with the teachings of the present invention.

FIG. 13C illustrates one aspect of the tape comprising a plurality of pills sealed within a package that can be coiled up and inserted inside a chamber of the apparatus of FIG. 13A, in accordance with the teachings of the present invention.

FIG. 13D illustrates one aspect of the medication delivery apparatus of FIG. 13A for delivering an individual pill from a tape comprising a plurality of pills sealed therein, in accordance with the teachings of the present invention.

FIG. 13E is a top view of a tape delivery mechanism for liquid medicine delivery of one aspect of the medication delivery apparatus of FIG. 13A, in accordance with the teachings of the present invention.

FIG. 13F is a side view of a tape delivery mechanism for liquid medicine delivery of one aspect of the medication delivery apparatus of FIG. 13A, in accordance with the teachings of the present invention.

FIG. 14A is an illustrative example of a user wearing a detector and making contact with an apparatus, which is shown in block diagram form, which delivers a medicinal dose in accordance with the teachings of the present invention.

FIG. 14B illustrates one aspect of the medication delivery apparatus of FIG. 14A for delivering an individual pill from a tape comprising a plurality of pills sealed therein while also delivering a dose of liquid, in accordance with the teachings of the present invention.

FIG. 15A is an illustrative example of a user wearing a detector and making contact with an apparatus, which is shown in block diagram form, which delivers a medicinal dose in accordance with the teachings of the present invention.

FIG. 15B illustrates one aspect of the medication delivery apparatus of FIG. 15A for delivering an individual does of liquid medication from a bladder, in accordance with the teachings of the present invention.

### DETAILED DESCRIPTION

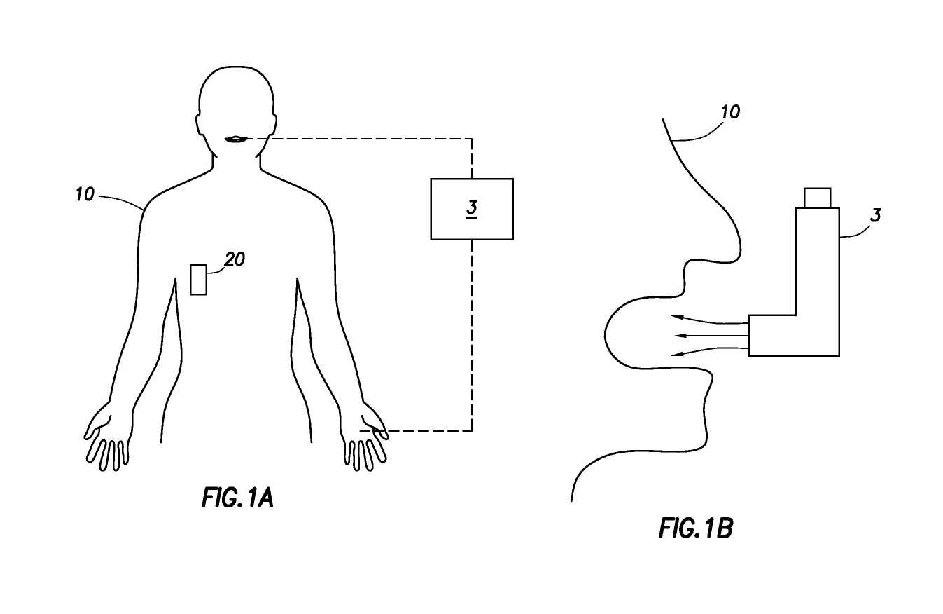

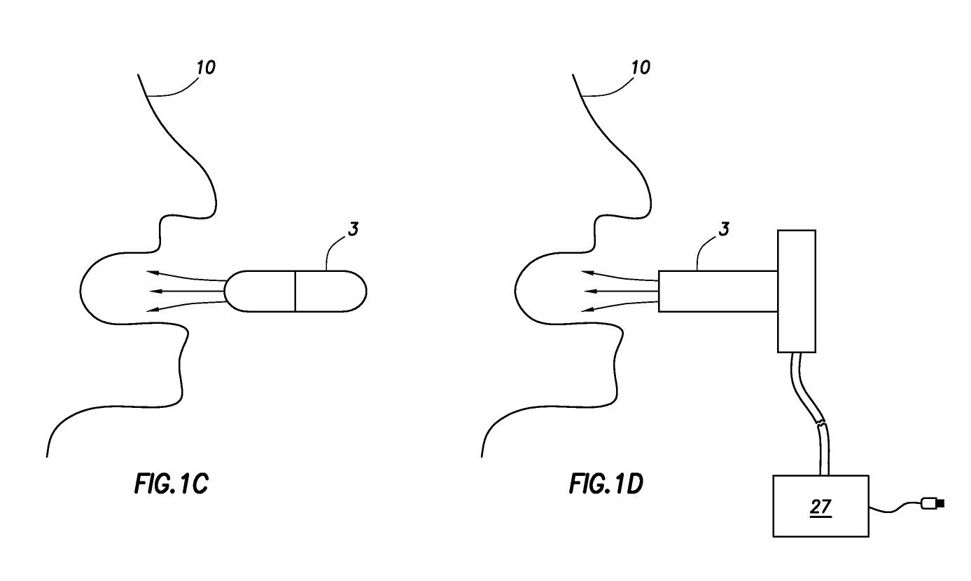

Referring to FIG. 1A, a user 10 is shown wearing a detector 20 and making physical contact with an apparatus 3; the detector 20 and the apparatus 3 are described in greater detail hereinbelow. The detector 20 is shown secured at one location on the user's body and is communicatively coupled to the user 10 and is capable of detecting a current flow through the user's body. However, the scope of various aspects of the present invention is not limited by the positioning of the detector 20 on the user's body. The detector 20 may be secured to any location on the user's body. In accordance with another aspect of the present invention, the detector 20 is secured to the user's clothing. In accordance with yet another aspect of the present invention, the detector 20 may be worn by the user in the form of jewelry, watch, apparel, etc. In such aspects, the detector 20 may be communicatively coupled to the user, e.g., contacting the user, in near physical proximity to the user, in communicative proximity to the user, etc.

In the present illustrated example, the detector 20 is shown as a detector that is external to the user's body. In accordance with another aspect of the present invention, the detector 20 may be positioned or implanted within the user's body. In yet another aspect of the present invention, the detector 20 may be partially implanted within the user's body. The externally secured detector 20 of interest includes those that are sized to be stably associated with a living subject in a manner that does not substantially impact movement of the living subject. As such, the detector 20 may have dimensions that, when secured to the user 10, will not cause the user 10 to experience any difference in mobility or movement.

In accordance with some aspects of the present invention, the detector 20 is dimensioned such that its size does not hinder the ability of the subject to physically move. For example, in one aspect the detector 20 has a small size and may occupy a volume of space of 5 cm³ or less, such as 3 cm³ or less, including 1 cm³ or less. In another aspect the detector 20 has a small size and may occupy a volume of space of 25 cm³ or less, such as 12 cm³ or less, including 5 cm³ or less. In another aspect the detector 20 has a small size and may occupy a volume of space of 25 cm³ or less, such as 5 cm³ or less, including 1 cm³ or less. In accordance with one aspect of the present invention a receiver (not shown) can be included in the apparatus 3. In such instances, the receiver has a chip size limit ranging from 2 mm² to 2 cm².

The user 10 holds the apparatus 3 in the user's hand and places the apparatus 3 to the user's mouth, thereby making contact with the apparatus 3 in at least two locations, as shown. In the case of a small child, the parent may hold the apparatus 3. In various aspects, if the child makes contact with the parent, then as described hereinbelow, a circuit is completed through the parent and child. The apparatus 3 is shown in block diagram form for clarity of describing the functionality thereof as well as the components therein. However, the apparatus 3 can be made is a variety of shapes according to the various aspects of the present invention.

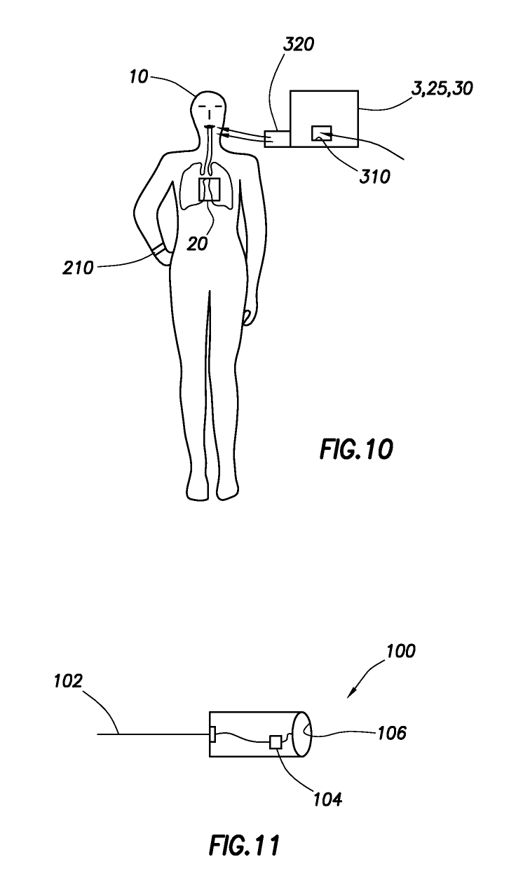

Referring now to FIGS. 1B, 1C, and 1D, in accordance with various aspects of the present invention, the apparatus 3 can be in a variety of shapes. For example, FIG. 1B shows the apparatus 3 as an upright inhaler with a chamber that is capable of receiving a pressurized can that contains multiple and/or single doses, each dose delivered individually over a period of time to the user 10. FIG. 10 shows the apparatus 3 as a single dose inhaler with a chamber that is capable of holding one dose. After the single dose is delivered to the user 10, the chamber is reloaded. FIG. 1D shows the apparatus 3 as an inhaler capable of providing continuous delivery of a dose or delivery of a dose at pre-determined intervals using a motorized unit 27 that is associated with a power source. Additional examples and shapes for the delivery of an inhalable dose are considered to be within the scope of the present invention. Thus, according to various aspects of the present invention, the apparatus 3 can be in various shapes and dose delivery set-ups, as shown in FIGS. 1B, 1C, and 1D and the scope of the present invention is not limited by the actual shape or dose-delivery type or dose-delivery timing of device.

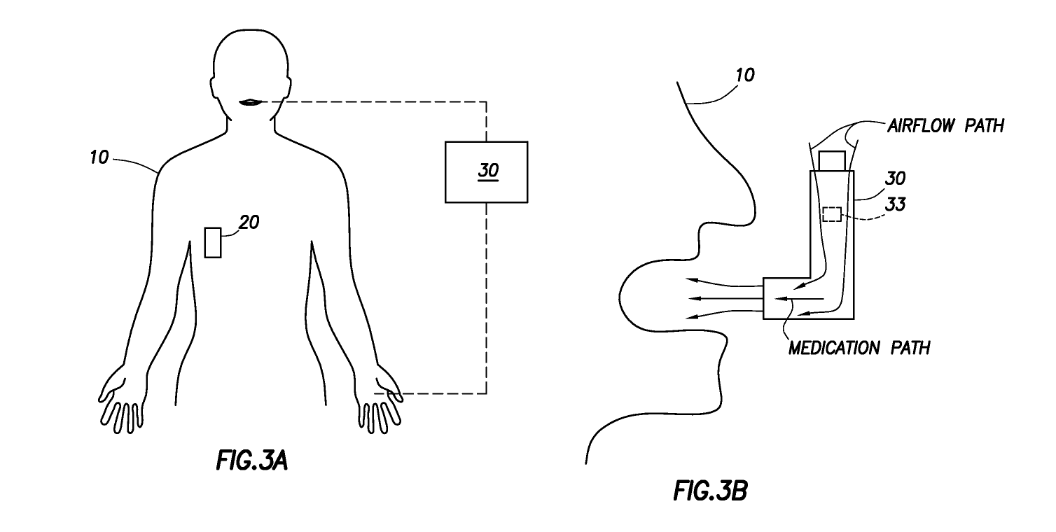

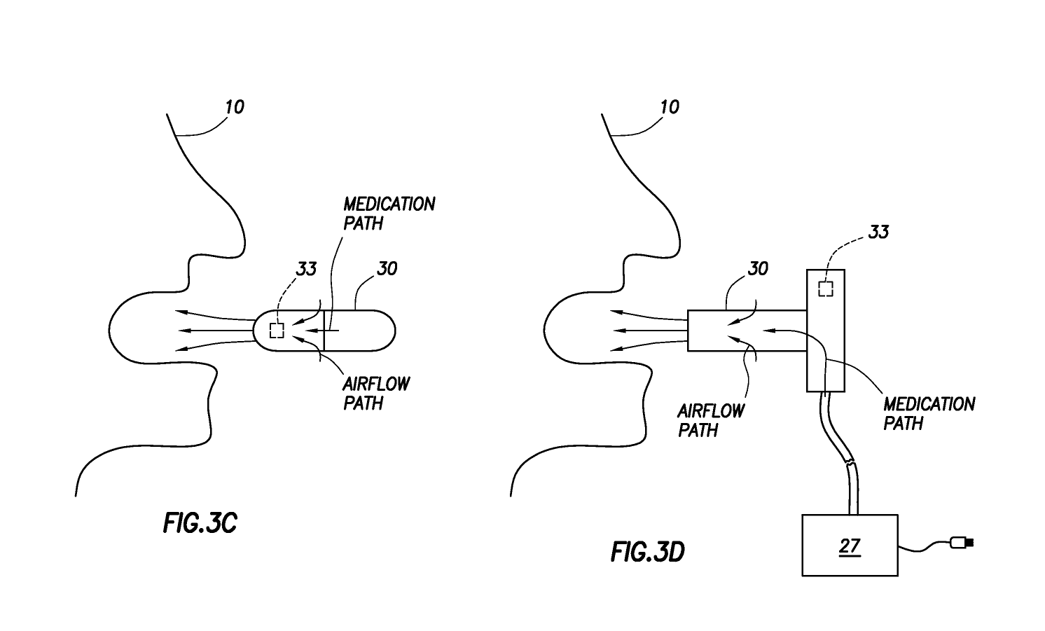

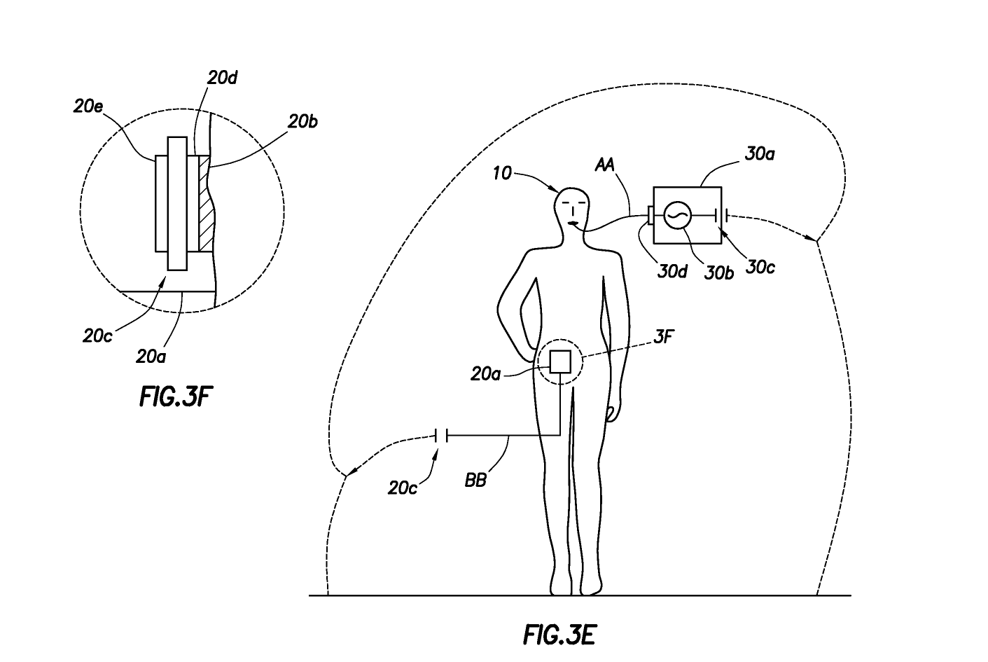

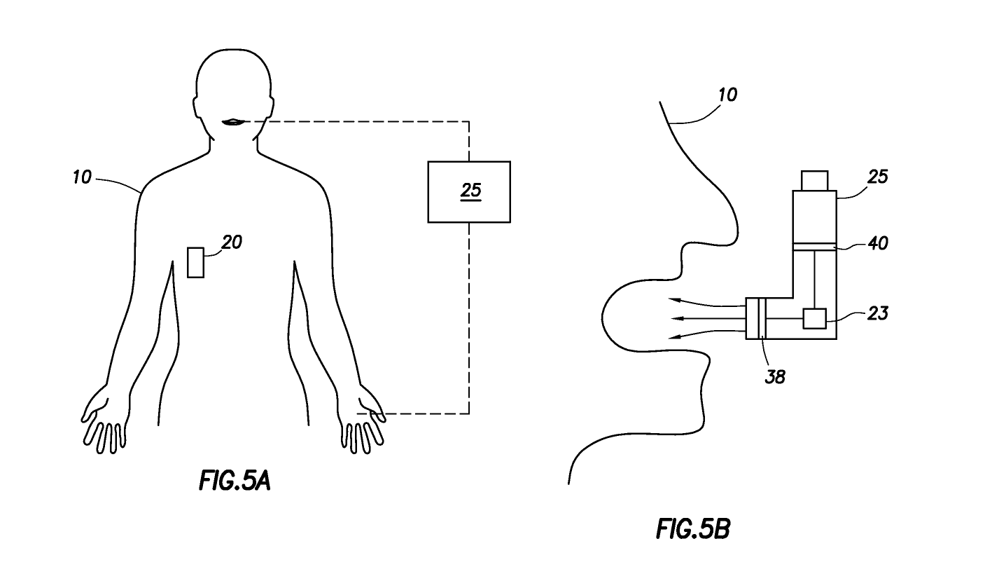

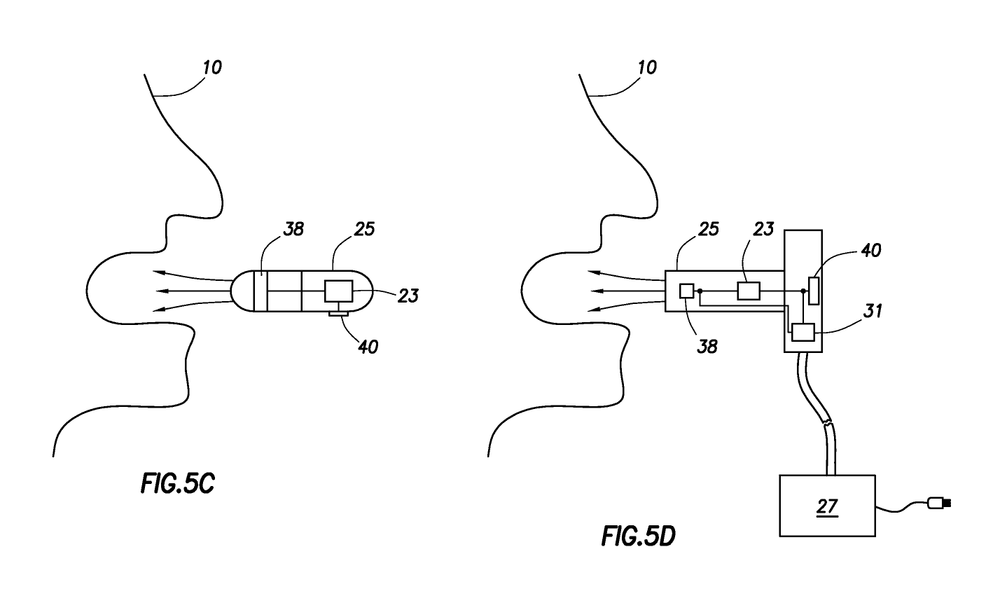

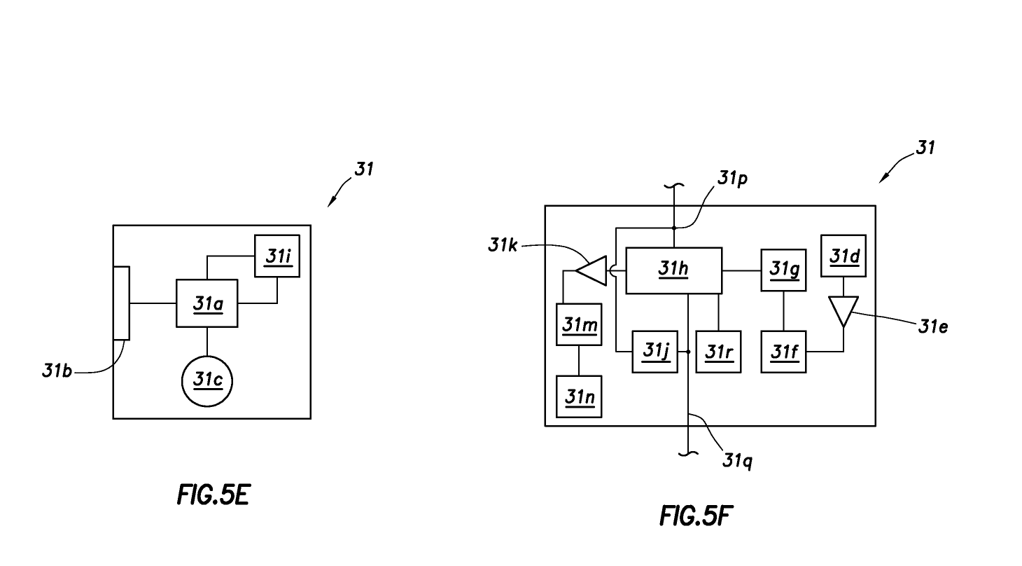

In various aspects, the example apparatus 3, may include an acoustic unit or acoustic unit in accordance with various aspects of the present invention as shown, for example, in connection with apparatus 30 and apparatus 25 as shown in FIGS. 3B and 5B, respectively, and as described in more detail hereinbelow.

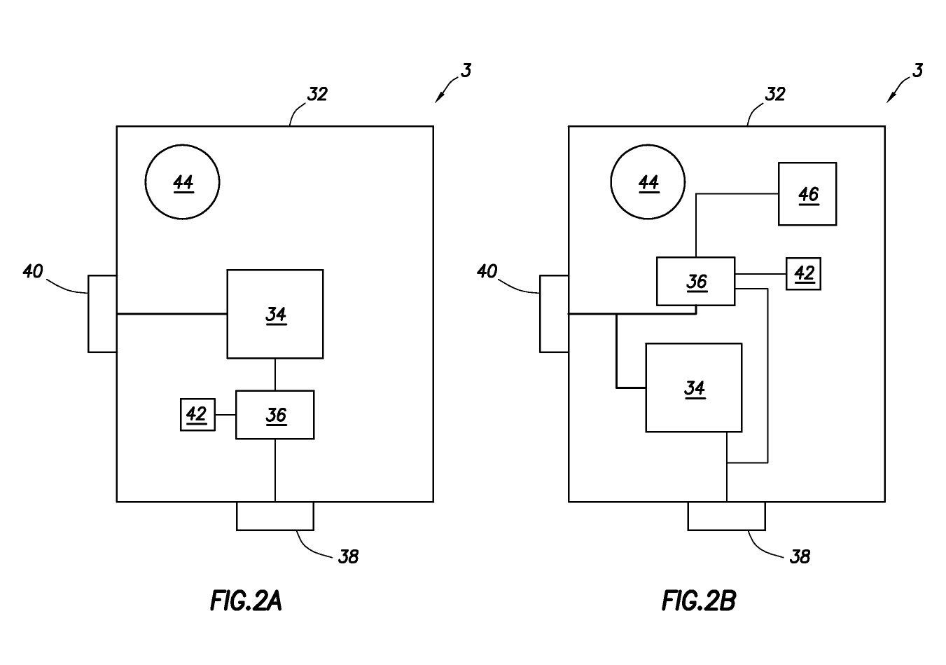

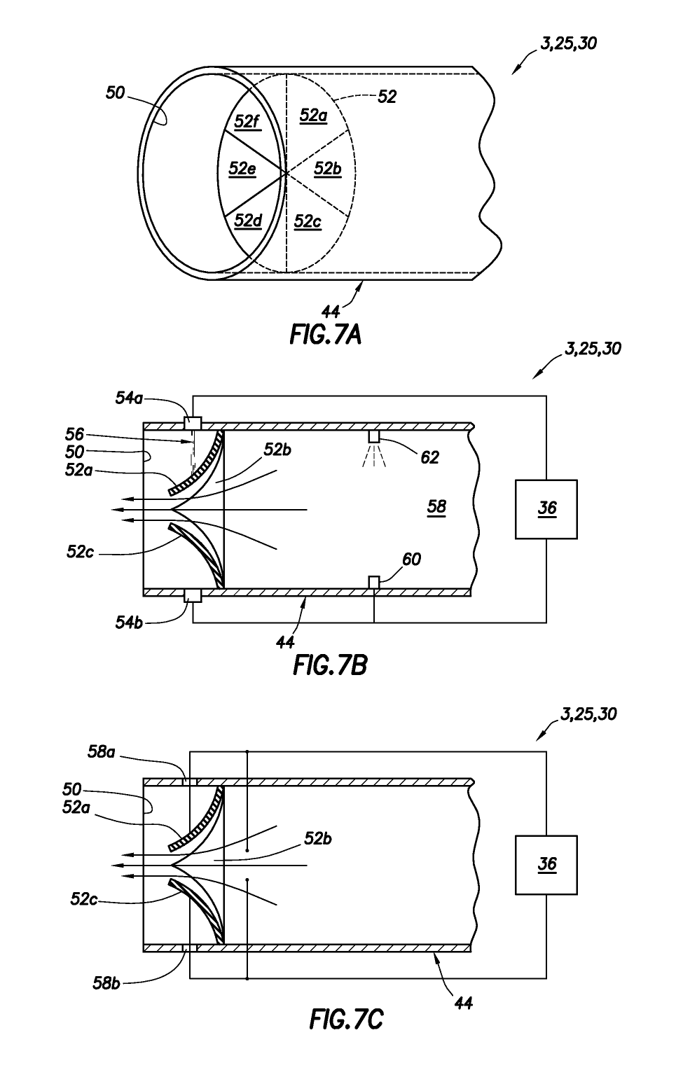

Referring now to FIG. 2A, the apparatus 3 includes a housing 32, a power source 34, a control unit 36, contact areas 38, 40 and a memory unit 42. The housing 32 defines a chamber 44 for holding the inhalable doses. The chamber 44 may also include a chamber control unit for controlling dispensing of the dose. The chamber 44 also includes an opening through which the inhalable dose is delivered to the patient, as discussed with respect to FIG. 7A hereinbelow. In accordance with one aspect of the present invention, the housing 32 includes at least two contact areas/points 38, 40 positioned at different locations on the housing 32. The location and position of the contact points 38, 40 or areas are determined by the shape and design of the apparatus 3 and in accordance with the various aspects of the present invention. Multiple locations for the contact areas 38, 40 are contemplated.

In accordance with another aspect of the present invention, the contact areas 38, 40 may include functionality that allow for reading and recording of biometric information, such as finger print data. This information may be communicated to the apparatus 3 and stored therein for confirmation of the user's identity.

The contact areas 38, 40 are electrically isolated from each other and at least partially exposed or exposable to allow the user 10 to make contact therewith. In one aspect, the contact areas 38, 40 may also be coated with a thin dielectric, such as plastic, and the electrical communication is accomplished using capacitive coupling through this dielectric to the user. Capacitive coupling may be accomplished at signal frequencies greater than about 20 kHz and in some aspects about 80 kHz for good signal-to-noise ratio (SNR, e.g., signal to ambient noise). In accordance with one aspect of the present invention, the contact areas 38, 40 are positioned such that one contact area 38, for example, makes contact with the user's hand and the other contact area 40, for example, makes contact with the user's mouth. In accordance with alternative aspect of the present invention, additional contact areas may be added to allow for secondary contacts with the hand or mouth as well as to accommodate using a different hand, such as a left hand grip as well or a right hand grip. Furthermore, additional contact areas on the housing 32 can be included to ensure that the apparatus 3 is held properly. If the contact areas 38, 40 are covered by dielectric, then the apparatus makes contact with the mouth of the user, and the electrodes may or may not be visible to the user. For example, the electrodes could be embedded in the plastic and sense the presence of the mouth using capacitive coupling and looking for a large change in impedance between the two capacitive plates that occurs when the mouth contact both of these plates. It will be appreciated that the contact areas 38, 40 may be located near or on the exterior of the housing 32. In one aspect, the contact areas 38, 40 may be “partially exposed” and in another aspect may be “embedded” in the housing 32. In other aspects, the user may couple to the contact areas 38, 40 using noncontact means such as detection changes of an electric field when user is in proximity to the contact areas 38, 40.

The power source 34 is electrically connected to the control unit 36 and coupled to the contact areas 38, 40. One terminal of the power source 34 is electrically connected to the contact area 40. The other terminal of the power source 34 is electrically connected to the control unit 36. As shown, the power source 34 has two outputs; one output is electrically connected to the control unit 36 and the output is connected to the contact area 38. In this example, the control unit 36 is connected in series with the power source 34. However, the scope of the present invention is not limited by the relative circuit relationship between the control unit 36 and the power source 34. For example, the control unit 36 may be positioned in parallel with the power source 34, such that the control unit 36 is electrically connected to both of the contact areas 38, 40 as well as both terminals of the power source 34. It will be appreciated that in certain aspects, the power source 34 produces alternating current (AC) signals so as not to fibrillate the heart. Other signals may be employed provided that safe operation for living subjects is considered.

As the apparatus 3 is brought into contact with the user's hand and mouth, the contact areas 38, 40 come into contact with the user 10 resulting in a complete circuit that includes the user 10. Thus, the circuit that defines the current path includes the user 10, the contact area 40, the power source 34, the control unit 36, and the contact area 38. Once the circuit path is completed the power source 34 provides the voltage potential needed to cause a current flow through the user's body. The presence of the current flow is an indicator that the apparatus 3 is in position for dispensing the inhalable dose because the apparatus 3 is now in the user's hand and has made contact with the user's mouth. The detector 20 identifies the presence of the current flow through the user's body and can record the timing of the occurrence of the event, as discussed in greater detail hereinbelow. Additionally, the apparatus 3 can also detect the presence of the current flow and record the time the event occurred. Thus, once the circuit is complete and the current flow is detected, then the control unit 36 can also record the timing of the completed circuit in the memory unit 42.

In accordance with another aspect of the present invention, the control unit 36 provides additional control functionality. According to one aspect, the control unit 36 can control the conductance of the circuit, which is completed through the user's body, to encode information in the current flow. In addition, the control unit may superimpose information that is stored in the memory in the current flow as well. For example, the timing of the completion of the circuit, information about the dose, or additional identifying information can be encoded in the current flow. The control unit 36 alters the conductance of the circuit. The altered conductance results in an alteration of the characteristics of the current flow. It is the altered characteristics that contain the information and, thus, the information is encoded in the current flow, as disclosed in U.S. patent application Ser. No. 12/564,017 entitled COMMUNICATION SYSTEM WITH PARTIAL POWER SOURCE filed on Sep. 21, 2009, and published as US 2010/0081894, the entire specification of which is incorporated herein by reference. The encoded information is then detected by the detector 20 and decoded. In addition, information disclosed in U.S. patent application Ser. No. 11/912,475 entitled PHARMA-INFORMATICS SYSTEM filed on Apr. 28, 2006, and published as US 2008/0284599 the entire specification of which is incorporated herein by reference.

Additionally, in accordance with another aspect of the present invention the control unit 36 may enter a sleep state to minimize power consumption. The control unit may return to an active state by any one or more known methods that involve electric power, heat, pressure, sound, etc. In cases when sound is used, sound vibrations generated by the dose being loaded into the chamber 44 may be used to generate an activation signal that is send from an acoustic unit, for example, to the control unit 36. The activation signal may be used to place the control unit 36 in an active state from the sleep state. Once activated, the control unit 36 can record additional acoustic information, especially information associated with the user 10 inhaling through the apparatus 3.

Referring now to FIG. 2B, in accordance with another aspect of the present invention, the control unit 36 can also control communication using additional or alternative communication channels, such as wireless and optical. The control unit 36 is coupled to and in communication with a transceiver 46 that is included in an alternative aspect of the apparatus 3. The transceiver 46 is configured to transmit and/or receive communications from the apparatus 3. Although the communication is described in terms of transmissions from the transmitter 46, in accordance with one aspect of the present invention a receiver (not shown) can be included in the apparatus 3 and electrically connected to the control unit 36. Furthermore and in accordance with another aspect of the present invention, the transmitter 46 may be replaced with a transceiver unit that handles both transmission and reception of information. Thus, the control unit 36 allows the apparatus 3 to communicate with the detector 20 using multiple communication channels and methods. In one aspect, the transceiver is configured to communicate with an external device such as a cell phone or a personal computer to indicated that the inhalation event occurred. The wireless communication may be instead of or in addition to generating a signal that indicates the user has inhaled.

The apparatus 3 can communicate with various other devices such as a computer with a built-in or peripheral monitor (such as may be found in a bedside monitor or a health information system), a personal digital assistant (PDA), a smart phone, a messaging device, a data center, etc. Additionally, the apparatus 3 may be configured to be communicatively coupled to, e.g., interrogated by, an external device to provide and/or receive data to an external location. Any convenient data transmission protocol may be employed, including both conduction through a physical medium (for example, through the user's body using the current flow) and through the air, such as wireless data transmission protocols.

In accordance with another aspect of the present invention, the detector 20 communicates identification code or information to the apparatus 3. The identification code or information activates the apparatus 3 to deliver the dose. Additionally, the detector 20 can confirm delivery of the dose and send confirmation information to the apparatus 3 indicating that the dose was delivered.

In accordance with yet another aspect of the present invention, both the detector 20 and the apparatus 3 communicate directly and independently with a third device, such as a cell phone or a computer. The information communicated is related to the delivery of the dose. This third device will then reconcile the data and information to correlate delivery time, dose amount, patient identity, and other related factors, each of which may be received from the detector 20 and/or the apparatus 3.

Referring still to FIG. 2B, the control unit 36 is positioned in parallel with the power source 34 because the control unit 36 is connected to each of the contact areas 38, 40. However, the scope of the present invention is not limited by the relative circuit position of the control unit 36 to the power source 34 within the circuit that is completed through the user's body. For example, the control unit 36 can be positioned in series with the power source 34 such that one output of the power source 34 is connected to the control unit 36 similar to that shown in FIG. 2A. As discussed above, the control unit 36 controls the conductance characteristics of the circuit and hence the characteristics of the current flow. In this manner the control unit 36 can encode information in the current flow and allow the current characteristics to carry information to the detector 20.

Referring now to FIG. 3A, the user 10 is shown wearing a detector 20 and making physical contact with an apparatus 30. The apparatus 30 is similar to the apparatus 3 described hereinbefore, and in various aspects the apparatus 30 includes an acoustic unit 33, as shown in FIGS. 3B-3D. For example, in FIG. 3B the acoustic unit 33 is shown in the path of the airflow but not in the path of the medication. In FIG. 3C the acoustic unit 33 is shown in the path of the airflow and the medication. In FIG. 3D the acoustic unit 33 is shown away from the path of the airflow and the medication. The scope of the present invention is not limited by the location of the acoustic unit 33 relative to the path of the airflow and medication flow. Various aspects of the detector 20 and the apparatus 30 are described in greater detail hereinbelow.

The user 10 holds the apparatus 30 in the user's hand and places the apparatus 30 to the user's mouth, thereby making contact with the apparatus 30 in at least two locations, as shown. In the case of a small child, the parent may hold the apparatus 30. The apparatus 30 is shown in block diagram form for clarity of describing the functionality thereof as well as the components therein. However, the apparatus 30 can be made is a variety of shapes according to the various aspects of the present invention.

Referring now to FIG. 3E, the user 10 is shown making contact through contact area AA to an apparatus 30a. The apparatus 30a is shown having a source 30b, a capacitive coupler 30c and a contact area 30d. The capacitive coupler 30c is electrically isolated from the contact area 30d. The contact area 30d is an electrode or plate that represents one side of a capacitor relative to ground. The user 10 is also shown wearing a detector 20a that includes a capacitive coupler 20c. A capacitive conductance path is shown with a broken line in FIG. 3E between the capacitive couplers 30c, 20c. In the figure, the physical contact between the user 10 and the apparatus 30a is shown by the line AA. It will be apparent that the contact between the user 10 and the apparatus 30a is actually a physical contact and it can occur between the mouth or hand of the user 10 and the apparatus 30a. The scope of the present invention is not limited by the area on the apparatus 30a that makes contact with the user 10. For example, any part of the user 10 that makes contact with the contact area 30d is within the scope of the present invention as disclosed in PCT Patent Application Serial No. PCT/2011/23017 entitled TWO-WRIST DATA GATHERING SYSTEM filed on Jan. 28, 2011, and in PCT Patent Application Serial No. PCT/2011/23013 entitled WRIST DATA GATHERING SYSTEM filed on Jan. 28, 2011, the entire specification each of which is incorporated herein by reference.

Referring now to FIG. 3E and FIG. 3F, the detector 20a is shown in an exploded view at a skin location 20b of the user 10. The capacitive coupler 20c includes a contact area 20d that is in contact or near contact with the user 10 at skin location 20b and a non-contact side 20e. Through the body of the user 10 and the contact area 30d, the source 30b is physically coupled to the contact side 20d. Line BB is shown to illustrate an electrical connection between a patch in accordance with one aspect of the present invention, through the air and/or ground, the capacitive coupler 30c is coupled to the capacitive coupler 20c of the detector 20a. Capacitive coupling occurs between the apparatus 30a and the detector 20a. Hence, in accordance with another aspect of the present invention, the apparatus 30a includes one contact area 30d that comes into contact with the patient while a second contact area, the capacitive coupling area 30c, which is isolated from the patient. The apparatus 30a uses a capacitive return path to the receiver via space or ground.

Thus, by controlling the isolating characteristics of the source 30a, using the control module of the apparatus 30a, information is encoded in the carrier wave by using known technologies. For example, using Frequency-shift Keying (FSK) information may be encoded in the carrier wave, including binary FSK.

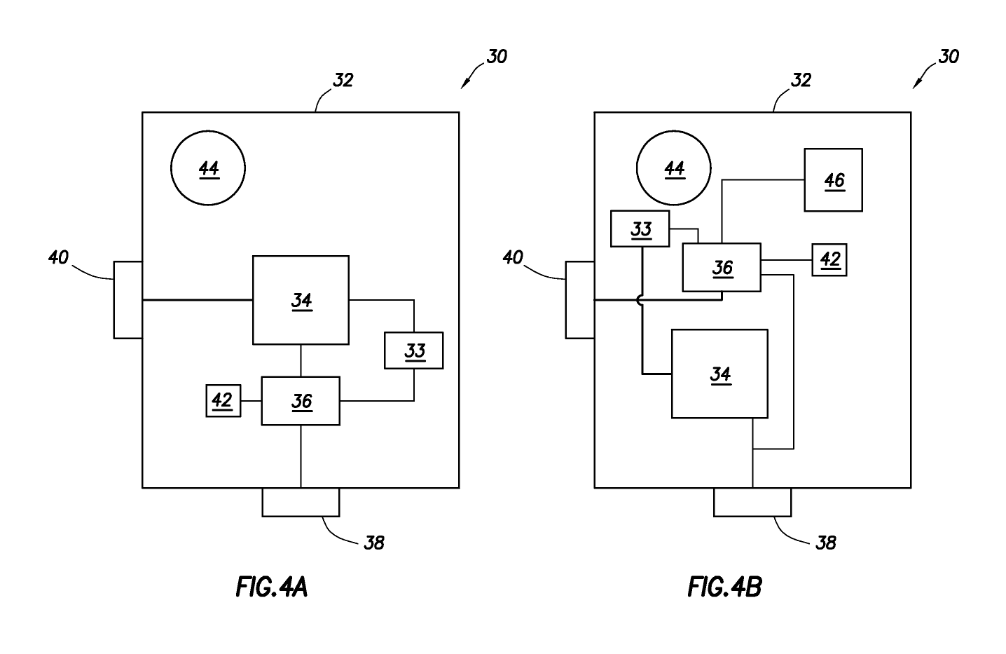

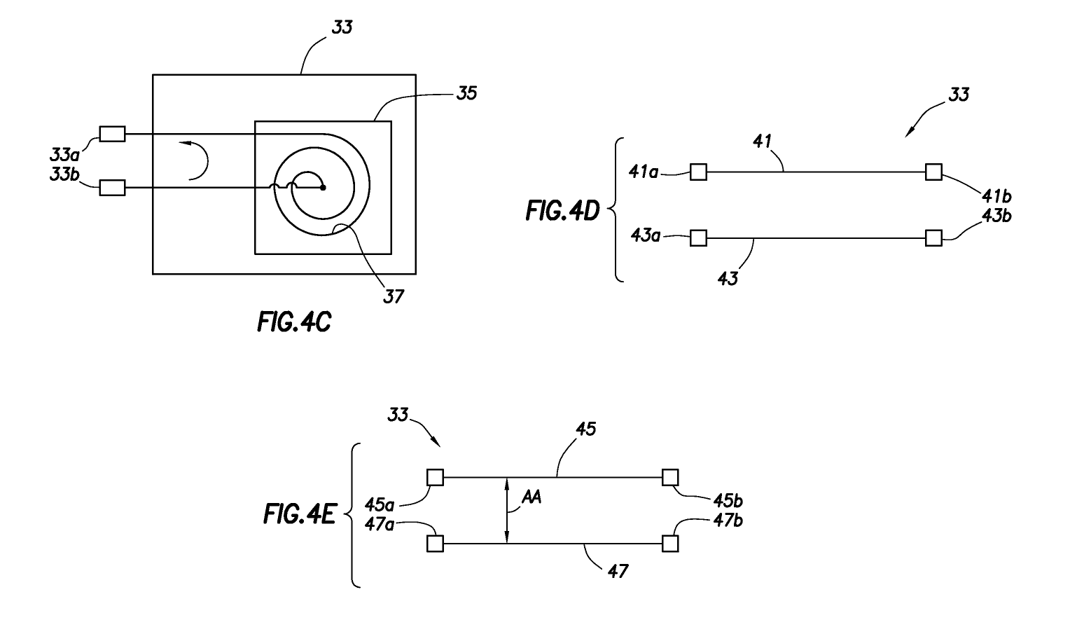

Referring now to FIG. 4A, the apparatus 30 includes a housing 32, the acoustic unit 33, a power source 34, a control unit 36, contact areas 38, 40 and a memory unit 42. The housing 32 defines a chamber 44 for holding the inhalable doses. The chamber 44 may also include a chamber control unit for controlling dispensing of the dose. The chamber 44 also includes an opening through which the inhalable dose is delivered to the patient, as discussed with respect to FIG. 7A hereinbelow. In accordance with one aspect of the present invention, the two contact areas/points 38, 40 may be positioned at different locations on the housing 32. The location and position of the contact points 38, 40 or areas are determined by the shape and design of the apparatus 30 and in accordance with the various aspects of the present invention. Multiple locations for the contact areas 38, 40 are contemplated.

The contact areas 38, 40 are electrically isolated from each other and at least partially exposed to allow the user 10 to make contact therewith. In accordance with one aspect of the present invention, the contact areas 38, 40 are positioned such that one contact area 38, for example, makes contact with the user's hand and the other contact area 40, for example, makes contact with the user's mouth. In accordance with alternative aspect of the present invention, additional contact areas may be added to allow for secondary contacts with the hand or mouth as well as to accommodate using a different hand, such as a left hand grip as well or a right hand grip. Furthermore, additional contact areas on the housing 32 can be included to ensure that the apparatus 30 is held properly.

In accordance with another aspect of the present invention, the contact areas 38, 40 may include functionality that allow for reading and recording of biometric information, such as finger print data. This information may be communicated to the apparatus 30 or the acoustic unit 33 and stored therein for confirmation of the user's identity.

The power source 34 is electrically connected to the acoustic unit 33, the control unit 36, and coupled to the contact areas 38, 40. The acoustic unit 33 is also electrically coupled to the control unit 36 and the operation thereof is discussed with respect to FIGS. 4C, 4D, and 4E. One terminal of the power source 34 is electrically connected to the contact area 40. The other terminal of the power source 34 is electrically connected to the control unit 36. As shown, the power source 34 has two outputs; one output is electrically connected to the control unit 36 and the other output is connected to the contact area 38. In this example, the control unit 36 is connected in series with the power source 34. However, the scope of the present invention is not limited by the relative circuit relationship between the control unit 36 and the power source 34. For example, the control unit 36 may be positioned in parallel with the power source 34, such that the control unit 36 is electrically connected to both of the contact areas 38, 40 as well as both terminals of the power source 34.

Additionally, in accordance with another aspect of the present invention the control unit 36 may enter a sleep state to minimize power consumption. In such a condition, any event causing a change of state may serve as an activation signal e.g., the sound vibrations generated by the dose being loaded into the chamber 44 may be used to generate an activation signal that is sent from the acoustic unit 33 to the control unit 36. The activation signal may be used to place the control unit 36 in an active state from the sleep state. Once activated, the control unit 36 can record additional acoustic information, especially information associated with the user 10 inhaling through the apparatus 30.

As the apparatus 30 is brought into contact with the user's hand and mouth, the contact areas 38, 40 come into contact with the user 10 resulting in a complete circuit that includes the user 10. Thus, the circuit that defines the current path includes the user 10, the contact area 40, the power source 34, the control unit 36, and the contact area 38. Once the circuit path is completed the power source 34 provides the voltage potential needed to cause a current flow through the user's body. The presence of the current flow is an indicator that the apparatus 30 is in position for dispensing the inhalable dose because the apparatus 30 is now in the user's hand and has made contact with the user's mouth. The detector 20 identifies the presence of the current flow through the user's body and can record the timing of the event, which is discussed in greater detail hereinbelow. Additionally, the apparatus 30 can also detect the presence of the current flow and record the time the event occurred. Thus, once the circuit is complete and the current flow is detected, then the control unit 36 can also record the timing of the completed circuit in the memory unit 42.

Referring now to FIG. 4B, in accordance with another aspect of the present invention, the control unit 36 can also control communication using additional or alternative communication channels, such as wireless and optical. The control unit 36 is coupled to and in communication with a transceiver 46 that is included in an alternative aspect of the apparatus 30. The transceiver 46 is configured to transmit and/or receive communications from the apparatus 30. Although the communication is described in terms of transmissions from the transmitter 46, in accordance with one aspect of the present invention a receiver (not shown) can be included in the apparatus 30 and electrically connected to the control unit 36. Furthermore and in accordance with another aspect of the present invention, the transmitter 46 may be replaced with a transceiver unit that handles both transmission and reception of information. Thus, the control unit 36 allows the apparatus 30 to communicate with the detector 20 using multiple communication channels and methods. In one aspect, the transceiver is configured to communicate with an external device such as a cell phone or a personal computer to indicated that the inhalation event occurred. The wireless communication may be instead of or in addition to generating a signal that indicates the user has inhaled.

The apparatus 30 can communicate with various other devices such as a computer with a built-in or peripheral monitor (such as may be found in a bedside monitor or a health information system), a PDA, a smart phone, a messaging device, a data center, etc. Additionally, the apparatus 30 may be configured to be interrogated by an external device to provide data to an external location. Any convenient data transmission protocol may be employed, including both conduction through a physical medium (for example, through the user's body using the current flow) and through the air, such as wireless data transmission protocols.

In accordance with another aspect of the present invention, the detector 20 communicates identification code or information to the apparatus 30. The identification code or information activates the apparatus 30 to deliver the dose. Additionally, the detector 20 can confirm delivery of the dose and send confirmation information to the apparatus 30 indicating that the dose was delivered.

In accordance with yet another aspect of the present invention, both the detector 20 and the apparatus 30 communicate directly and independently with a third device, such as a cell phone or a computer. The information communicated is related to the delivery of the dose. This third device will then reconcile the data and information to correlate delivery time, dose amount, patient identity, and other related factors, each of which may be received from the detector 20 and/or the apparatus 30.

Referring still to FIG. 4B, the control unit 36 is positioned in parallel with the power source 34 because the control unit 36 is connected to each of the contact areas 38, 40. However, the scope of the present invention is not limited by the relative circuit position of the control unit 36 to the power source 34 within the circuit that is completed through the user's body. For example, the control unit 36 can be positioned in series with the power source 34 such that one output of the power source 34 is connected to the control unit 36 similar to that shown in FIG. 4A. As discussed above, the control unit 36 controls the conductance characteristics of the circuit and hence the characteristics of the current flow. In this manner the control unit 36 can encode information in the current path and allow the current characteristics to carry information to the detector 20.

Referring now to FIG. 4C, in one aspect the acoustic unit 33 includes a magnet 35 and a coil 37. The coil 37 is connected to the control unit 36 of FIG. 4A through connections 33a, 33b. The coil 37 is secured to or mounted on a movable or flexible surface that is positioned proximal to the magnet 35. The control unit 36 measures the current generated as the coil 37 moves within or through the magnetic field associated with the magnet 35. As the relative position of the coil 37 to the magnet 35 changes, the change in distance results in a change in the characteristic of the magnetic field. Thus, sound waves produced by inhaling or loading the medication for delivery, results in movement of the coil 37 within the magnetic field of the magnet 35. Thus, sound waves can be detected by the control unit 36 through the coil 37.

Referring now to FIG. 4D, the acoustic unit 33 is shown in accordance with another aspect of the present invention to include a fixed coil 41 and a movable coil 43. The fixed coil 41 is connected to the power source 34 through connection point 41a and 41b. The power supplied to the fixed coil 41 results in a magnetic field about the fixed coil 41. The movable coil 43 is positioned proximal to the fixed coil 41 and connected to the control unit 36 through the connection points 43a, 43b. The control unit 36 detects the presence of the magnetic field associated with the fixed coil 41 through the movable coil 43. As the relative position of the movable coil 43 to the fixed coil 41 changes, the change in distance results in a change in the characteristic of the electromagnetic field associated with the fixed coil 41. As the user 10 dispenses the medication into or inhales through the apparatus 30, the resulting sound waves result in movement of the movable coil 43. Thus, sound waves produced by inhaling or loading the medication for delivery, results in movement of the movable coil 43 within the electromagnetic field of the fixed coil 41. Thus, sound waves can be detected by the control unit 36 through the movable coil 43.

Referring now to FIG. 4E, the acoustic unit 33 is shown in accordance with another aspect of the present invention to include a fixed plate 45 and a movable plate 47. The fixed plate 45 is connected to the power source 34 through connection point 45a and 45b. The power supplied to the fixed plate 45 results in a buildup of a charge about the fixed plate 45. The movable plate 47 is positioned proximal to the fixed plate 45 and connected to the control unit 36 through the connection points 47a, 47b. The control unit 36 measures the capacitive coupling between the fixed plate 45 and the movable plate 47 through the movable coil 43. As the position of the movable plate 47 relative to the fixed plate 45 changes, the change in distance results in a change in the characteristic of the capacitance associated with the gap AA between the fixed plate 43 and the movable plate 47. As the user 10 dispensed the medication into or inhales through the apparatus 30, the resulting sound waves reach the movable plate 47. Thus, sound waves produced by inhaling or loading the medication for delivery, results in movement of the movable plate 47. Accordingly, sound waves can be detected by the control unit 36 through the movable coil 43.

One advantage is that the acoustic sensors of the present invention may be fabricated at a low cost using manufacturing techniques borrowed from printed circuit board (PCB) fabrication—either etching or additive/printing. Further advantage may be gained by incorporating the circuits associated with the controller unit, which are in accordance with the various aspects of the present invention, onto the “circuit” board using additive/printing techniques. Furthermore, in accordance with another aspect of the present invention, the power source may be incorporated into the assembly.

In accordance with various aspects of the present invention, the acoustic unit 33, as indicated hereinabove, may be positioned in various locations within the apparatus 30 (FIG. 3A), such as out of both the airflow path and the medication path as shown in FIG. 3D, within the airflow path and out of the medication path as shown in FIG. 3B, or within both the airflow path and the medication path as shown in FIG. 3C. Additionally, in accordance with another aspect of the present invention, the acoustic unit 33 may be positioned internal or external to the apparatus 30.

Referring now to FIG. 5A, a user 10 is shown wearing a detector 20 and making physical contact with a medication delivery apparatus 25; the detector 20 and the apparatus 25 are described in greater detail hereinbelow.

The user 10 holds the apparatus 25 in the user's hand and places the apparatus 25 to the user's mouth, thereby making contact with the apparatus 25 in at least two locations, as shown. In the case of a small child, the parent may hold the apparatus 25. If the child makes contact with the parent, then as described hereinbelow, a circuit is completed through the parent and the child. The apparatus 25 defines a chamber for holding the inhalable doses. The chamber may also include a chamber control unit for controlling dispensing of the dose. The chamber also includes an opening through which the inhalable dose is delivered to the patient, as discussed with respect to FIG. 7A hereinbelow.

Referring now to FIGS. 5B, 5C, and 5D, in accordance with various aspects of the present invention, a dose confirmation apparatus 23 is secured to the apparatus 25, which can be in a variety of shapes. In accordance with one aspect of the device, the dose confirmation apparatus 23 includes an acoustic unit 31. For example, FIG. 5B shows the dose confirmation apparatus 23 secured to the apparatus 25 and the contact areas 38, 40. The dose confirmation apparatus 23 includes the acoustic unit 31. The apparatus 25 is an upright inhaler with a chamber that is capable of receiving a pressurized can that contains multiple, or single doses, where each dose is delivered individually over a period of time to the user 10. FIG. 5C shows the apparatus 25 as a single dose inhaler with a chamber that is capable of holding one dose. After the single dose is delivered to the user 10, the chamber is reloaded.

In accordance with another aspect of the present invention, the dose confirmation apparatus 23 and the acoustic unit 31 are independent units that can be individually secured to the apparatus 25. For example, referring to FIG. 5D, the dose confirmation apparatus 23 and the acoustic unit 31 are independent and each secured to the apparatus 25. The apparatus 23 and the acoustic unit 31 are in communication with each other and connected to the contact areas 38, 40. The apparatus 25 is an inhaler capable of providing continuous delivery of a dose or delivery of a dose at pre-determined intervals using a motorized unit 27 that is plugged into a power source. The power source disclosed in accordance with aspects of the present invention may be an AC power supply or a direct current (DC) power supply. Furthermore, the power supply is designed to ensure that the current flow operates within safe parameters for living subjects.

Additional examples and shapes for the delivery of an inhalable dose are considered and within the scope of the present invention. Thus, according to various aspects of the present invention, the apparatus 25 can be in various shapes and dose delivery set-ups, as shown in FIGS. 5B, 5C, and 5D and the scope of the present invention is not limited by the actual shape or dose-delivery type or dose-delivery timing of the apparatus 25.

Furthermore, the scope of the present invention is not limited by the inclusion of the acoustic unit 31 within the apparatus 25 or the separation of the acoustic unit 31 from the apparatus 25. More specifically, each have specific functionality and purpose and as such operate independent of the other as well as in cooperation with each other. Thus, any device, including any commonly known or conventional inhaler, can be used with either or both the apparatus 25 and/or the acoustic unit 31 to confirm delivery of a dose to a user 10 without structural changes or modification to the apparatus 25.

In accordance with one aspect of the present invention, the acoustic unit 31 includes an aperture that generates sound as the user 10 inhales. The sound is generated by the airflow through the acoustic unit 31, in a manner similar to a whistle. The acoustic unit 31 is placed in the path of the airflow. As the user 10 inhales, the airflow through the apparatus 25 and the acoustic unit 31 generates an acoustic signal.

Referring now to FIG. 5E, in accordance with another aspect of the present invention the acoustic signal generated by the acoustic unit 31 is an electrically generated signal in response to detecting the user 10 inhaling. The acoustic unit 31 includes a processing unit 31a that includes a memory unit, a sound generation unit 31b, a vibration detection unit 31c, and a power source 31i. As the user 10 inhales through the apparatus 25, the vibration detection unit 31c detects the vibration and produces a signal in response to the detection event. The signal is sent to the processing unit 31a to indicate that the user 10 has inhaled. The processing unit 31a signals the sound generation unit 31b to produce an acoustic signal. Furthermore, the vibration unit 31c can also detect the vibration produced by the device as the dose is loaded into a chamber, as discussed below. The vibrations associated with the loading of the dose and the user 10 inhaling, each produces a unique vibration signature that can be used independently or collectively to confirm delivery of the dose to the user 10. Furthermore, in accordance with another aspect of the present invention the acoustic unit 31 can communicate through a transceiver with an external device such as a cell phone or a personal computer, as discussed herein, to indicated that the inhalation event occurred. The wireless communication may be instead of or in addition to generating a signal, such as, for example, the acoustic signal that indicates the user has inhaled.

Referring now to FIG. 5F, in accordance with another aspect of the present invention, the acoustic unit 31 includes a power source 31j and a microphone 31d. The microphone 31d detects sound vibrations generated by inhaling and/or loading of the dose in the chamber of apparatus 25 for delivery. Thus, the event of loading the dose with intent to deliver as well as the actual event of inhaling at the time of delivery can be independently detected. The detected sound is passed through an amplifier 31e and a filter 31f to a digitizer or an analog-to-digital converter 31g. The filter 31f, in accordance with one aspect of the present invention, is a low pass filter. The digitizer 31g provides a digital signal to a controller 31h. The controller 31h may be a programmable processor or microprocessor wherein the control instructions can be programmed and reprogrammed.

The range of acoustic information generated by the loading of the dose and the inhaling event has typically known characteristics. Thus, the acoustic information captured or collected by the microphone 31d has known characteristics. For example, a particular device may produce a unique sound as the dose is loaded or as the user 10 inhales through the apparatus 25. Given the known range of acoustic information or sound produced by each device as the dose is loaded and the unique characteristics of the sound produced as the user 10 inhales, a state machine or a finite state machine can be used instead of a microprocessor. Thus, in accordance with one aspect of the present invention, the controller 31h is a state or a finite state machine.

The controller 31h provides a signal, which passes through an amplifier 31k and a filter 31m, to a sound generation unit or speaker 31n that produces an acoustic signal. The signal sent from the controller 31h to the speaker 31n includes any one or more of the following types of data: identification information that is unique to the acoustic unit 31 to identify the type of medication or dose delivered; information associated with the timing of the dose being loaded into the apparatus 25; and the timing of the user 10 inhaling. This information is then transmitted from the acoustic unit 31 to the detector 20 using the acoustic signal.

The acoustic signal generated by the acoustic unit 31 is detected, as described below, by the detector 20 secured to the user 10. In this way, any device, including any commonly known or conventional inhaler, can be used with the acoustic unit 31 to confirm delivery of a dose to the user 10 without structural changes or modification to the device used to deliver the dose. As indicated above, in accordance with another aspect of the present invention the acoustic unit 31 can communicate through a transceiver to indicate that the inhalation event has occurred.

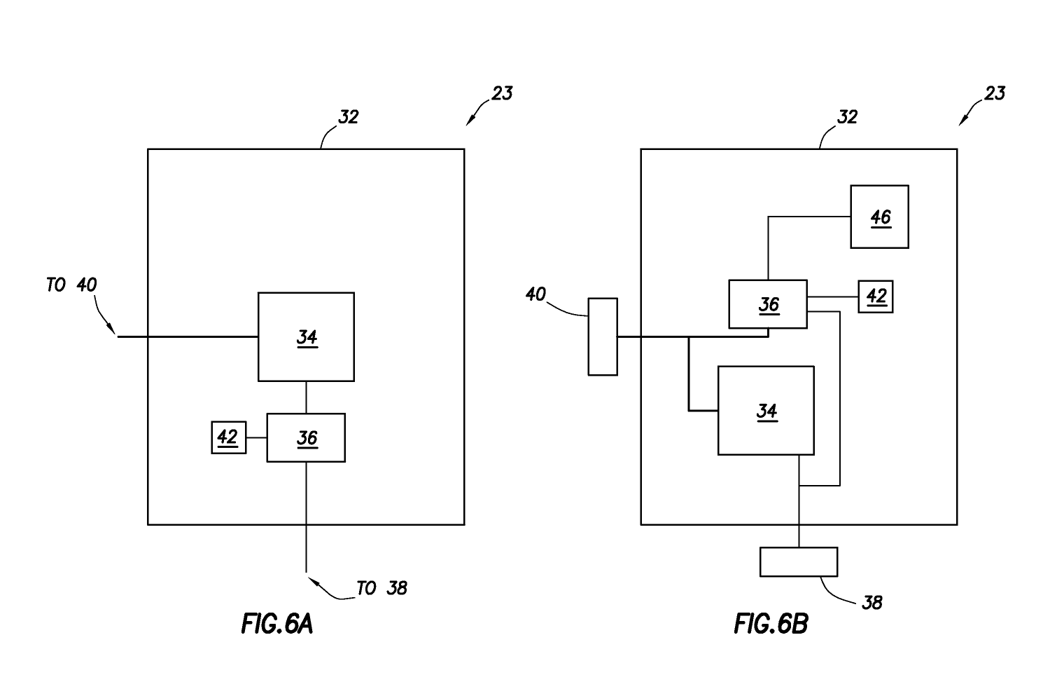

In accordance with another aspect of the present invention, the acoustic unit 31 also includes contact connections 31p and 31q. The contact connections 31p and 31q are electrically coupled to or connected to contact areas 38 and 40, which are described with respect to FIG. 6A hereinbelow.

The information gathered by the controller 31h, such as physiological or environmental information, is stored in the memory unit 31r for later retrieval and aggregation.

In accordance with another aspect of the present invention, the acoustic unit 31 includes a wireless communication module, such as a Bluetooth® module, that communicates using ultra low power consumption with an external device. For example, the external device may be any commercially available hardware/software device such as a cell phone, a computer, a network router, a telemedicine base station, as well as embedded technology such as a single chip device, a transceiver, and a key fob. Thus, the scope of the present invention is not limited by the type of device that communicates with the acoustic unit 31. Similarly, the scope of the present invention is not limited by the type of device that communicates with the apparatus 25. Furthermore, the data exchanged includes firmware images, raw collected data, generated information, data management information, and configuration data.

Referring now to FIG. 6A, the dose confirmation apparatus 23 is shown in block diagram form for clarity of describing the functionality thereof as well as the components therein. However, the dose confirmation apparatus 23 can be made is a variety of shapes according to the specific design needs and the shape of the apparatus 25. The dose confirmation apparatus 23 includes a power source 34, a control unit 36, contact areas 38, 40 and a memory unit 42. In accordance with one aspect of the present invention, the housing 32 includes at least two contact areas/points 38, 40 positioned at different locations on the housing 32. The location and position of the contact points 38, 40 or areas are determined by the shape and design of the apparatus 25 and in accordance with the various aspects of the present invention. Multiple locations for the contact areas 38, 40 are contemplated. Furthermore, the contact area 38, 40 may be part of the apparatus 25 or part of the dose confirmation apparatus 23 or part of the acoustic unit 31. In accordance with another aspect of present invention, the contact areas 38, 40 may be separate and independently attached to the apparatus 25 to allow customization of the contact areas 38, 40 relative to the location of the dose confirmation apparatus 23 or the acoustic unit 31 on the apparatus 25.

In accordance with another aspect of the present invention, the contact areas 38, 40 may include functionality that allow for reading and recording of biometric information, such as finger print data. This information may be communicated to the dose confirmation apparatus 23 or the acoustic unit 31 and stored therein for confirmation of the user's identity.

As the apparatus 25 is brought into contact with the user's hand and mouth, the contact areas 38, 40 come into contact with the user resulting in a complete circuit that includes the user 10. Thus, the circuit that defines the current path includes the user, the contact area 40, the power source 34, the control unit 36, and the contact area 38. Once the circuit path is completed the power source 34 provides the voltage potential needed to cause a current flow through the user's body. The presence of the current flow is an indicator that the apparatus 25 is in position for dispensing the inhalable dose because the apparatus 25 is now in the user's hand and has made contact with the user's mouth. The detector 20 identifies the presence of the current flow through the user's body and can record the timing of the event, which is discussed in greater detail below. Thus, once the circuit is complete and the current flow is detected, then the control unit 36 can also record the timing of the completed circuit in the memory unit 42.

Referring again to FIGS. 5E and 5F, in accordance with various aspects of the present invention, the processing unit 31a and the controller 31h can perform the function of the control unit 36.

In accordance with another aspect of the present invention, the control unit 36 provides additional control functionality. According to one aspect, the control unit 36 can control the conductance of the circuit, which is completed through the user's body, to encode information in the current flow. For example, the timing of the completion of the circuit, information about the dose, or additional identifying information can be encoded in the current flow. The control unit 36 alters the conductance of the circuit. The altered conductance results in an alteration of the characteristics of the current flow. It is the altered characteristics that contain the information and, thus, the information is encoded in the current flow, as disclosed in U.S. patent application Ser. No. 12/564,017 entitled COMMUNICATION SYSTEM WITH PARTIAL POWER SOURCE filed on Sep. 21, 2009, the entire specification of which is incorporated herein by reference. The encoded information is then detected by the detector 20 and decoded.

Additionally, in accordance with another aspect of the present invention the control unit 36 may enter a sleep state to minimize power consumption. In such a condition, the sound vibrations generated by the dose being loaded into the chamber 44 may be used to generate an activation signal that is send from the acoustic unit 31 (FIG. 5D) to the control unit 36. The activation signal may be used to place the control unit 36 in an active state from the sleep state. Once activated, the control unit 36 can record additional acoustic information, especially information associated with the user 10 inhaling through the apparatus 25.

Referring now to FIG. 6B, in accordance with another aspect of the present invention, the control unit 36 can also control communication using additional or alternative communication channels, such as wireless and optical. The control unit 36 is coupled to and in communication with a transmitter 46 that is included in an alternative aspect of the dose confirmation apparatus 23. Although the communication is described in terms of transmissions from the transmitter 46, in accordance with one aspect of the present invention a receiver (not shown) can be included in the dose confirmation apparatus 23 and electrically connected to the control unit 36. Furthermore and in accordance with another aspect of the present invention, the transmitter 46 may be replaced with a transceiver unit that handles both transmission and reception of information. Thus, the control unit 36 allows the dose confirmation apparatus 23 to communicate with the detector 20 using multiple communication channels and methods. In one aspect, the transceiver is configured to communicate with an external device such as a cell phone or a personal computer to indicated that the inhalation event occurred. The wireless communication may be instead of or in addition to generating a signal that indicates the user has inhaled

The dose confirmation apparatus 23 can communicate with various other devices such as a computer with a built-in or peripheral monitor (such as may be found in a bedside monitor or a health information system), a PDA, a smart phone, a messaging device, a data center, etc. Additionally, the dose confirmation apparatus 23 may be configured to be interrogated by an external device to provide data to an external location. Any convenient data transmission protocol may be employed, including both conduction through a physical medium (for example, through the user's body using the current flow) and through the air, such as wireless data transmission protocols.

Referring again to FIG. 6B, the control unit 36 is positioned in parallel with the power source 34 because the control unit 36 is connected to each of the contact areas 38, 40. However, the scope of the present invention is not limited by the relative circuit position of the control unit 36 to the power source 34 within the circuit that is completed through the user's body. For example, the control unit 36 can be positioned in series with the power source 34 such that one output of the power source 34 is connected to the control unit 36 similar to that shown in FIG. 6A. As discussed above, the control unit 36 controls the conductance characteristics of the circuit and hence the characteristics of the current flow. In this manner the control unit 36 can encode information in the current path and allow the current characteristics to carry information to the detector 20.

Referring again to FIG. 5A, the detector 20 is shown secured to the subject at one location. The location of the detector 20 is be determined by the medical requirements and the system. The detector 20 employed in accordance with the various aspects of the present invention is configured to be associated with a body location (either inside, partially inside of, or on a surface of a body) and to detect current and electrical signals from one or more devices, such as the apparatus 25 of FIG. 5A. It is also within the scope of the present invention to have the detector 20 attached to the clothing of the subject with just electrode leads/wires secured to, or otherwise in contact with, the skin of the subject.

Referring now to FIG. 7A, in accordance with one aspect of the present invention the apparatus 3, 30, 25 (as described in connection with FIGS. 1A-6B hereinabove) is shown with an opening 50 and a diaphragm 52. The diaphragm 52 is shown with six portions 52a, 52b, 52c, 52d, 52e, and 52f. The diaphragm 52 is positioned between the chamber 44 of the apparatus 3, 30, 25 and the opening 50. Thus, as the dose is being dispensed and the user 10 (FIGS. 1A, 3A, 5A) inhales, the portions 52a-f of the diaphragm 52 flex in the direction of the opening 50 and are separated to allow the dose to travel from the chamber 44 through the opening 50 to the user 10.

Referring now to FIG. 7B, in accordance with one aspect of the present invention, the apparatus 3, 25, 30 (as described in connection with FIGS. 1A-6B hereinabove) includes optical beam sensors 54a and 54b positioned between the diaphragm 52 and the opening 50. As shown, when the diaphragm portions 52a, 52b, and 52c are flexed to allow passage of the dose (as indicated by the direction of the arrows) from the chamber 44 through the opening 50 to the user 10 (FIGS. 1A, 3A, 5A), the optical beam 56 between the sensors 54a and 54b is interrupted. The interruption or breaking of the beam 56 is caused by the portions 52a-f of the diaphragm. This event is an indication that the dose is being inhaled by the user 10 and the sensors 54a and 54b send a signal to the control unit 36 of the apparatus 3, 25, 30. The control unit 36 can then either encode the information in the current flow or transmit this information to the detector 20 and thereby confirm that the dose was delivered to the user 10.

In accordance with another aspect of the present invention, the chamber 44 may also include an optical gap 58 prior to the diaphragm 52. In the optical gap, at least one optical sensor 60 is positioned to detect an optical emission from a light source 62 also included in the optical gap, such as a Light Emitting Diode (LED). As the dose is released into the optical gap of the chamber 44, the optical sensor 60 detects a drop in the intensity of the light due to the dose being present, which is a powder-like opaque substance. The optical sensor 60 is electrically coupled to the control unit 36. The optical sensor 60 signals the control unit 36 to indicate the presence of the dose once a change in optical intensity is detected.

Referring now to FIG. 7C, in accordance with one aspect of the present invention, the apparatus 3, 25, 30 (as described in connection with FIGS. 1A-6B hereinabove) includes impedance measurement units 58a and 58b positioned between the diaphragm 52 and the opening 50. In the closed position, the diaphragm 52 has a unique impedance when the portions 52a-f are in contact, which is measured by the impedance measurement units 58a and 58b. Once the diaphragm portions 52a-f are flexed and separated, the impedance characteristic of the diaphragm changes. As shown, when the diaphragm portions 52a, 52b, and 52c are flexed to allow passage of the dose (as indicated by the direction of the arrows) from the chamber 44 through the opening 50 to the user 10 (FIGS. 1A, 3A, 5A), the movement of the diaphragm portions 52a, 52b, and 52c causes a change in impedance of the diaphragm 52. This change in impedance is detected by the impedance measurement units 58a and 58b and is communicated to or signaled to the control unit 36 of the apparatus 3, 25, 30. The control unit 36 can then either encode the information in the current flow or transmit this information to the detector 20 and thereby confirm that the dose was delivered to the user 10.

In accordance with another aspect of the present invention, the device 20 includes a capacitive tactile sensor control unit (not shown). Once the device 20 comes into contact with the user's hand and mouth, then the control unit 36 receives a signal from the sensor control unit and the control module activates a chamber control unit. The chamber control unit initiates dispensing of the dose. According to one aspect to the present invention, the device 20 continues to dispense the inhalable dose to the user 10 (FIGS. 1A, 3A, 5A) such that the device 20 is dispensing a continuous dose. According to another aspect of the present invention, the device 20 dispenses a single dose. Once the sensor control unit detects that the device 20 is no longer in contact with the mouth or hand of the user, then the sensor control unit sends a deactivate or second signal to the control unit 36. The control unit 36 in turn signals the chamber control unit to stop dispensing the dose.

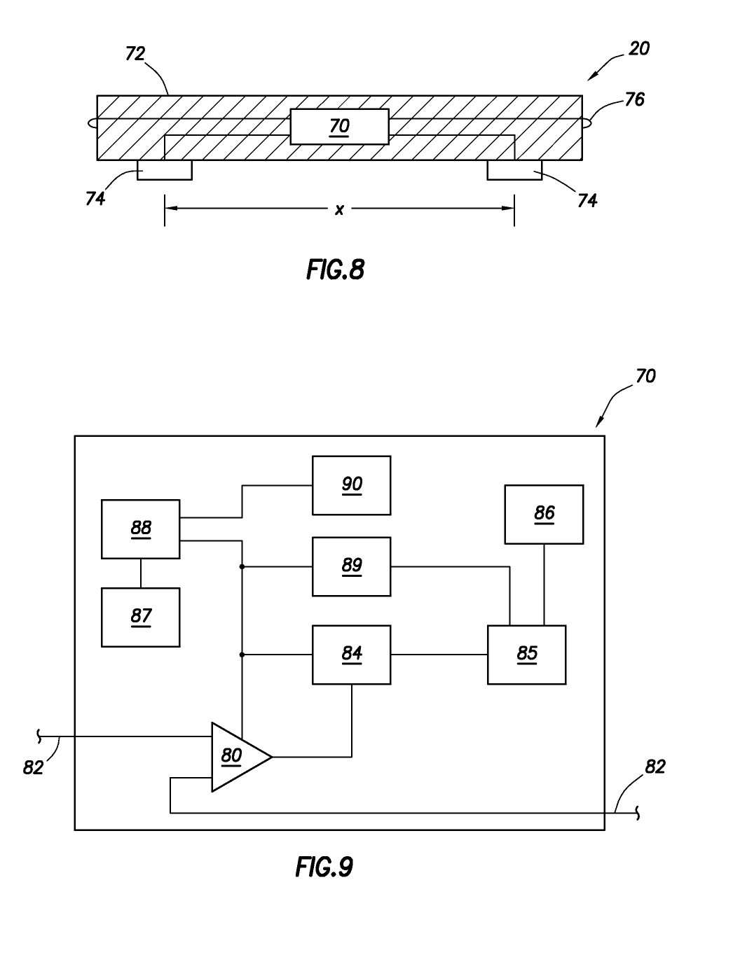

Referring now to FIG. 8, the detector 20 includes a processing unit 70 positioned in a housing 72. The processing unit 70 is electrically coupled to and connected to partially exposed electrodes 74. A coil 76 is wrapped around the housing 72 and electrically coupled to the processing unit 70. The coil 76 is wound around the perimeter and acts as an antenna for signal transmission and reception by the detector 20. In the current example, the detector 20 includes two electrodes. However, in accordance with another aspect of the present invention, the detector 20 may include fewer or greater electrodes and the scope of the present invention is not limited by the number electrodes associated with the detector 20. Thus, in one configuration according an aspect of the present invention, the detector 20 includes one or more electrodes (such as two or more electrodes, three or more electrodes, and/or includes multiple pairs of electrodes) for detecting the current signature traveling through the user's body from the apparatus 3, 25, 30 (as described in connection with FIGS. 1A-7C hereinabove). In one configuration of interest, the detector 20 includes two electrodes that are dispersed at a distance “X” from each other, which distance may be one that allows the electrodes to detect a differential voltage potential. This distance may vary, and may range from 0.1 to 5 cm, such as from 0.5 to 2.5 cm. The detector 20 may include a variety of different types of signal receiver elements and processing protocols. Additionally, the detector 20 may be either external to the user's body or implantable as disclosed in U.S. patent application Ser. No. 12/564,017 entitled BODY-ASSOCIATED RECEIVER AND METHOD filed on Feb. 12, 2010, and published as 2010-0312188 A1, incorporated herein by reference in its entirety.

Referring now to FIG. 9, the processing unit 70 includes an amplifier 80 that detects the differential voltage potential across the electrodes 74 of FIG. 8. The detected voltage potential, which represents the current characteristics, is sent to the amplifier 80 through leads 82 that are electrically connected to the electrodes 74 via the amplifier 80. The detected current characteristics then go into the demodulator 84. Also shown is a memory unit 85 coupled to the demodulator 84, a clock 86, and a transceiver unit 89. The memory unit 85 is capable of storing information, including data associated with the delivery of the dose to the user 10 as well as changes in the user's physiological condition after the dose is delivered to or inhaled by the user 10. The clock 86 provides timing information and writes to the memory unit 85 in order to timestamp the events that are recorded in the memory unit 85. The transceiver unit 89 transfers data from the memory unit 85 to an external data processor unit, not shown. For example, the transceiver unit 89 can receive information from an external device or communicate directly through the internet to collect information about environmental parameters as well as provide specific information including: physiological parameters about the patient; detection of potential events (e.g., episodes or attacks) as indicated by changes in heart rate, body temperature, respiration rate, and level, duration and timing of physical activity; timing of delivery of dose; frequency of dose delivery; and changes in physiological parameters in response to the dose delivery. Thus, the detector 20 can provide information, through a wireless communication, to automate detection of adverse events before delivery and reaction to medication after delivery. Furthermore, the detector 20 can use environmental parameters, such as pollen count or air pollution levels, to provide specific information to the user regarding potential for possible events. Furthermore, the detector 20 can record and provide baseline data associated with the user 10. For example, with a baseline measure of heart rate and respiration relative to the level of physical activity, the onset of an attack can be detected if there is a high rate of respiration with limited or no physical activity.

The processing unit 70 also includes a power source 87 electrically coupled to a microprocessor 88. The microprocessor 88 is electrically coupled to all the components and coordinates the function between the various functional blocks as well as power management. In accordance with another aspect of the present invention, the components of the processing unit 70, including the microprocessor 88, may be all connected to a bus and, hence, interconnected electrically to each other through the bus and controlled by the microprocessor.

According to other aspects of the present invention, the overall system that includes the detector 20 and the apparatus 3, 25, 30 (as described in connection with FIGS. 1A-7C hereinabove) communicate and each record a portion of the event associated with delivery of the dose to the user. For example, as shown in FIG. 9, the processing unit 70 includes a module 90. With the detector 20 positioned on the user's body proximal to the lungs, the act of inhaling can be detected by the module 90, wherein the module 90 is an accelerometer, and the event recorded.

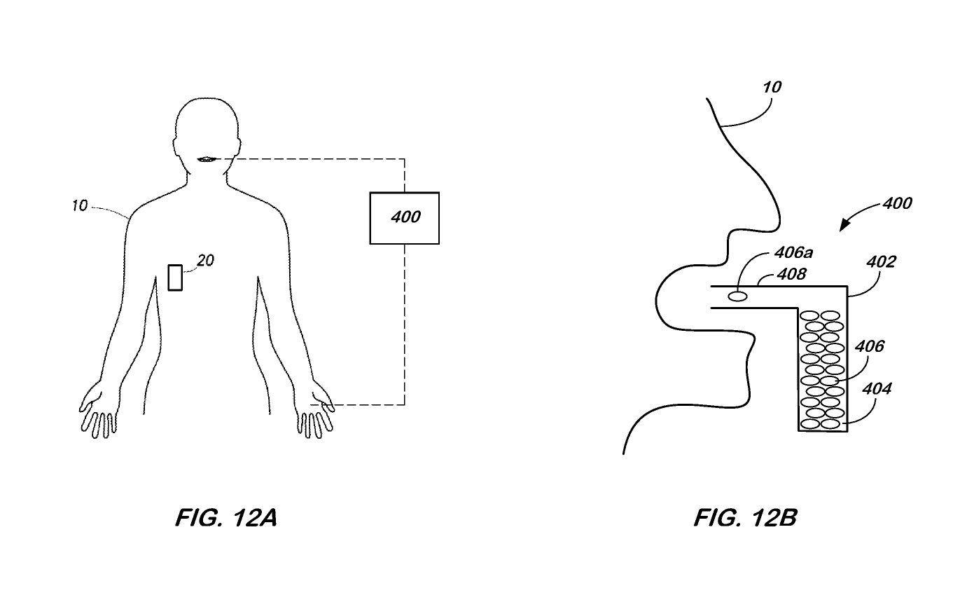

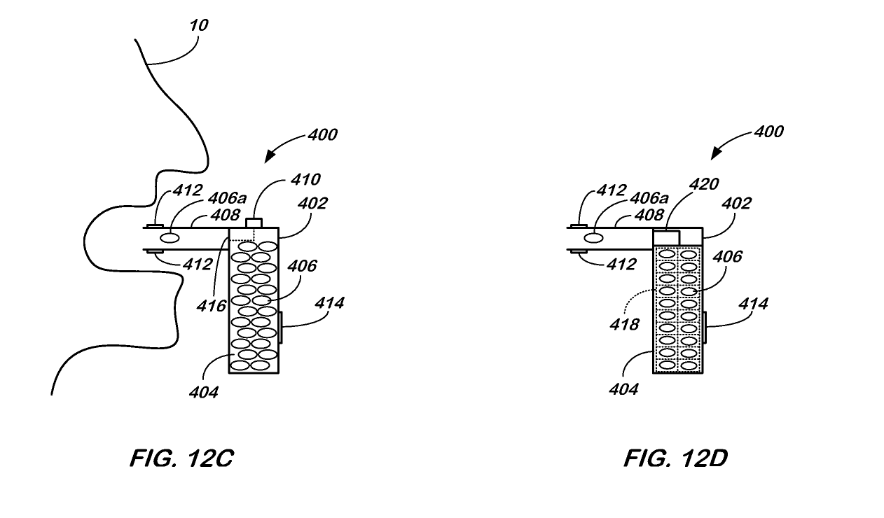

According to another aspect of the present invention, the processing unit 70 of the detector 20 wherein the module 90 is an acoustic detector. The acoustic detector 90 would detect the sound associated with the user 10 taking a deep breath. In accordance with another aspect of the present invention, the acoustic detector 90 also detects the sound or acoustic signal produced by the acoustic unit 33 of FIG. 4C or the acoustic unit 31 of FIG. 5E. If the detector 20 has detected the presence of the current flow from the apparatus 3, 25, 30 (as described in connection with FIGS. 1A-7C hereinabove and 400, 500, 600, 700 as described in connection with FIGS. 12A-15B hereinbelow) and records the sound associated with inhaling, and/or the sound associated with the dose being loaded as produced by the acoustic unit 33 of FIG. 4C or the acoustic unit 31 of FIG. 5E, then the acoustic detector 90 would record the event of inhalation as an indication of delivery of the dose to the patient.

According to another aspect of the present invention, the processing unit 70 is coupled to the module 90 that includes an optical detector. The optical detector of the module 90 would be positioned within the detector 20 such that a portion of the housing would be transparent and allowing for an optical beam to reach the optical detection unit 90.

According to another aspect of the present invention, the processor 70 of the detector 20 can include any combination of the accelerometer, the acoustic detector, and the optical detector.

According to various aspects of the present invention, the system of the invention may include a single detector or multiple detectors. For systems that include a single detector, the detector may include three or more distinct electrodes, and may be configured to be positioned in an abdominal or xyphoid region of the subject. The detector of such systems may be positioned at any convenient location, such as the front of a torso, the back of a torso, etc., as desired. In systems that have multiple detectors, each receiver may have a single electrode and such receivers may be in communication with one another to create an array of detectors.

In accordance with another aspect of the present invention, the acoustic detector and the current flow detection may occur within the apparatus 3, 25, 30 (as described in connection with FIGS. 1A-7C hereinabove) and the existence of both the current flow and the acoustic vibration would indicate that the device is being held in position by the user 10 (FIGS. 1A, 3A, 5A) and the dose has been loaded into the chamber.

In accordance with another aspect of the present invention, an activation signal may be generated from a mechanical action using a mechanical switch that is triggered as the dose is loaded in to the chamber or through a mechanical motion required by the inhaler to arm the dose delivery.

Referring now to FIG. 10, the user 10 places the apparatus 3, 25, 30 (as described in connection with FIGS. 1A, 3A, 5A) to his or her mouth and inhales. In accordance with another aspect of the present invention, the apparatus 3, 25, 30 defines an aperture 310. As the user 10 inhales through the mouth piece 320 of the apparatus 3, 25, 30, the flow of air through the aperture 310 produces a sound, which is similar to the sound produced by a whistle. These sound waves produced by the apparatus 3, 25, 30 travel through the surrounding air as well as into the lungs of the user 10. The sound waves enter the lungs of the user 10 and travel through the user's body. The detector 20 is secured to the user 10 and includes an acoustic detector that detects acoustic waves traveling through the air as well as through the body of the user 10. Thus, as the user 10 inhales, the sound waves produced by aperture 310 of the apparatus 3, 25, 30 can be detected and correlated with the event associated with deliver of the dose to the user 10. The combined detection of the sound wave through the air and tissue of the use is confirmation that the user 10 inhaled through the apparatus 3, 25, 30 and received the dose. Furthermore, in accordance with another aspect of the present invention, the aperture 310 of the apparatus 3, 25, 30 may be adjusted to create a specific or unique frequency that is further used as validation that the user 10 inhaled through the apparatus 3, 25, 30 as expected.

In accordance with another aspect of the present invention, a detector 210 may be used in the form of a watch worn on the wrist of the user 10.

In accordance with yet another aspect of the present invention, the detector may be implanted and capable of communicating with a detector located external to the body of the user. In this manner, acoustic waves traveling through the user's body may be detected and correlated with acoustic waves traveling external to the user's body.

Referring now to FIG. 11, an access device 100 is shown having a needle 102 and a data collection module 104 in accordance with another aspect of the present invention. The access device 100 defines an access port or opening 106. A user or patient in a hospital or outside of the hospital in home care situations very often has an access device 100, such as the access device 100, placed within the user for periods of time ranging from hours to many days, weeks or even months. The access device 100 is used for many purposes that include both the introduction and removal of fluid from the body. The access device 100 can be used to inject or infuse medication and basic fluids as well as removing fluids such as blood and waste. Typical examples of an access device 100 includes: Catheters (venous, urinary, etc,), IV Access ports, ostomy ports, etc.

With all of these devices there are high incidences of infections that result from general use and cleaning. The proper care of the access device 100 involves the proper cleaning and disinfecting of the port before it is used, especially for the access device 100 that remains in place for more than a single use. In its most basic form, the cleaning involves the act of wiping the access port 106 with a disinfectant like alcohol before inserting a syringe or other device into the access port 106. There are generally accepted good practices for this activity which involve proper technique and time spent with each activity. The data collection module 104 is coupled to the needle 102 and the rim of the access port 106. Information regarding the management, cleaning, and use of the access device 100 can be collect and stored by the data collection module 104. This information may be transmitted or communicated to a detector on the users' body, such as the detector 20 of FIGS. 1A, 3A, 5A. The information can be communicated to the detector using any of the disclosed communication protocols, including controlling the current signature or altering the carrier wave.

In accordance with various aspects of the present invention, the data collection module 104 can be added to the access device 100 as either an add-on device or a redesigned assess port that tracks the care of the port. For example, contact with the rim of the access port 106 can be detected and, hence, the time spent cleaning of the access port 106, including the amount of time spent wiping and the drying the cleaned access port 106 is recorded. Furthermore, the time of insertion of a syringe or other device into the port may be recorded as well as the amount of time the syringe spends in the access port 106. The collection of data can be used to show that the maintenance of the access port 106 had been done properly and in the proper sequence, and that it was done with each use of the port, as disclosed in U.S. Provisional Patent Application Ser. No. 61/377,072 entitled SYSTEM AND METHOD FOR PATIENT ACCESS PORT REPORTING filed on Aug. 25, 2010, incorporated herein by reference in its entirety.

In accordance with various aspects of the present invention, the data collection module 104 senses the cleaning or wiping of the access port 106 using a number of methods, including impedance measurement, acoustic measurement, and pressure measurement. For example, impendence measurement is achieved by first measuring the impedance between electrodes positioned at the access port 106 without the presence of a conduction fluid, such as the alcohol. As the access port is cleaned by alcohol or similar conducting fluid, the impedance between the electrodes is altered by the alcohol or disinfecting wipe and the change in impedance can be measured and the timing of the change can be recorded. As the alcohol evaporates and the wiping action is ended, the impedance returns to the previous level.

In accordance with another aspect of the present invention, acoustic detection devices may be used to detect the signature or vibrations associated with the wiping action. Additionally, the data collection module 104 can detect the pressure associated with the wiping action at the access port 106. The access device 100 makes an electrical connection to the patient to allow the information to be communicated to the detector using alteration of the current signature. In accordance with various aspects of the present invention, the electrical connection between the access device 100 necessary for communication between the data collection module 104 and the detector 20 worn by the user may be made physically or mechanically with any number of methods including: conductive tape, conductive or conductor imbedded tubing.

In accordance with another aspect of the present invention, the communication connection between the detector 20 worn by the user and the data collection module 104 of the access device 100 may be through capacitive conductance using the tubing wall or using the fluid in the tubing as the conductor.

In accordance with other aspects of the present invention, sensors on the access device 100 may be used to verify proper handling or holding of the access device 100 and to activate the data collection module 104 to document the cleaning and use of the access device 100. For example, the device could be designed to verify that the nurse was using gloves when handling and accessing the port.

In accordance with another aspect of the present invention, the data collection module 104 may also be used, through its contact with the user, to measure local skin/body temperature and send a signal to the detector, which would warn of infection.

Aspects of implantable versions of the detector may have a hermetically sealed and biologically compatible cavity defined to act as the enclosure, e.g., Chipskin™ technology one or more sensing electrodes, a power source, which could either be a primary cell or rechargeable battery, or one that is powered by broadcasting inductively to a coil. For the external signal receivers, aspects include structures that have electrodes in contact with the user's skin. The communication may be wireless or performed over one or more conductive media, e.g., wires, optical fibers, etc. Where desired, the same electrodes may be used for receiving and transmitting signals.

Having described various aspects of medication delivery systems for the delivery of an inhalable dose, the description now turns to various additional aspects of medication delivery systems for the delivery of medication products in a variety of forms generally. In various aspects such medication delivery systems can be adapted and configured to deliver medication products in the form of an ingestible product such as, for example, a pill, a tablet, a sub-lingual tablet, a polymeric dissolvable tablet, a capsule, a gel capsule, a time-release oral dosage, a suppository, a liquid, a liquid capsule, a liquid in combination with a pill, tablet (dissolvable or otherwise), or capsule, a blister pack containing a pill, tablet (dissolvable or otherwise), capsule, liquid, gel, among other forms of medication. In various aspects, the medication may include ingestible devices, such as, for example, ingestible event markers (IEMs) as disclosed in U.S. patent application Ser. No. 12/564,017 entitled COMMUNICATION SYSTEM WITH PARTIAL POWER SOURCE filed on Sep. 21, 2009, and published as US 2010/0081894, the entire specification of which is incorporated herein by reference. In another example, the medication may include ingestible devices comprising radio-frequency identification (RFID) functionality, e.g., active RFID devices or passive RFID devices. The medication may be sealed or unsealed depending on the particular configuration. The scope of the disclosed aspects, however, is not limited by the shape or type of product. An ingestible product along with the terms “ingested” or “ingest” or “ingesting” is understood to mean any introduction of the product internal to the body and may include oral, anal, vaginal introduction of the product or any introduction of the product into a natural body orifice.

Referring now to FIG. 12A, the user 10 is shown wearing a detector 20 and making physical contact with an apparatus 400. The apparatus 400 is configured for delivery of medication in the form of a pill, tablet, or capsule (pill). The detector 20 is shown secured at one location on the user's body and is communicatively coupled to the user 10 and is capable of detecting a current flow through the user's body. However, the scope of various aspects of the present invention is not limited by the positioning of the detector 20 on the user's body. The detector 20 may be secured to any location on the user's body. In accordance with another aspect of the present invention, the detector 20 is secured to the user's clothing. In accordance with yet another aspect of the present invention, the detector 20 may be worn by the user in the form of jewelry, watch, apparel, etc. In such aspects, the detector 20 may be communicatively coupled to the user, e.g., contacting the user, in near physical proximity to the user, in communicative proximity to the user, etc. In one aspect, the detector 20 may be positioned on the user's body so as to detect the movement of muscles associated with the user 10 swallowing a pill dose. Additional swallowing detection techniques are contemplated based on the positioning of electrodes or contact areas in various positions and orientations on the user's body.

As discussed hereinabove, the detector 20 may be located external to the user's body. In accordance with another aspect of the present invention, the detector 20 may be positioned or implanted within the user's body especially in connection with detection of the movement of the tongue associated with the act of swallowing. For example, during the act of swallowing, the tongue moves upwardly towards the palate. Accordingly, the detector 20 or contact areas in communication with the detector may be positioned to detect such movement to confirm the act of swallowing. In yet another aspect of the present invention, the detector 20 may be at least partially implanted within the user's body. The externally secured detector 20 of interest includes those that are sized to be stably associated with a living subject in a manner that does not substantially impact movement of the living subject. As such, the detector 20 may have dimensions that, when secured to the user 10, will not cause the user 10 to experience any difference in comfort, mobility or movement.

FIG. 12B illustrates one aspect of the medication delivery apparatus 400 for delivering an individual pill from a reservoir of pills. The apparatus 400 comprises a housing 402 having a chamber 404 (reservoir or container) portion for storing a plurality of individual pills 406 and a delivery tube 408 or device for delivering an individual pill dose 406a. The user 10 holds the apparatus 400 in the user's hand and places the apparatus 400 to the user's mouth, thereby making contact with the apparatus 400 in at least two locations. The user 10 then activates a pill delivery mechanism to deliver a prescribed pill dose 406a into the delivery tube 408. Although the apparatus 400 is shown delivering a single dose 406a, the apparatus may be adapted or configured to deliver multiple pill doses in accordance with a prescribed medication plan. In the case of a small child, the parent may hold the apparatus 400. In various aspects, if the child makes contact with the parent, then as described hereinbelow, a circuit is completed through the parent and child. The apparatus 400 is shown in block diagram form for clarity of describing the functionality thereof as well as the components therein. However, the apparatus 400 can be made is a variety of shapes according to the various aspects of the present invention. For example, in one aspect, the apparatus 400 can be adapted or configured to deliver a suppository in the user's rectum. Accordingly, the detector 20 would be positioned in a manner to be able to detect actual delivery of the suppository dose.

FIG. 12C illustrates one aspect of the medication delivery apparatus 400 for delivering an individual pill from a reservoir of pills. The apparatus 400 illustrated in FIG. 12C comprises a housing 402 having a chamber 404 (reservoir or container) portion for storing a plurality of individual pills 406 and a delivery tube 408 or device for delivering an individual pill dose 406a. The apparatus 400 further comprises an actuator 410 operatively coupled to a pill valve 416 port or latch mechanism for controlling the delivery of the individual pill dose 406a through the pill valve 416, which may be electrically, mechanically, or electromechanically enabled. In various aspects, the actuator 410 may comprise a mechanical button, lever, or spring coupled to the port 416 such that contacting the actuator by the user causes the valve 416 to deliver the single pill dose 406a. In other aspects, the actuator may comprise an electromechanical or electrical button or switch electronically coupled to the electrically enabled pill valve 416 such that contact with the actuator 410 results in the electronic control of the position of the port 416 or latch to deliver a single pill dose 406a. Other combinations of mechanical, electrical, electronic, and/or electromechanical actuating mechanisms are contemplated as well as other ports or latch mechanisms to control the delivery of a precise pill dose, which is generally one, but may be configured to control the delivery of multiple pill doses as well.

Also illustrated in FIG. 12C are at least two contact areas 412 and 414 that are electrically coupled to the detector 20 via the user's body. The user 10 holds the apparatus 400 in the user's hand and places the apparatus 400 to the user's mouth, thereby making contact with the first contact area 412 with the user's mouth and the second contact area 414 with the user's hand, thereby making contact with the apparatus 400 in at least two locations, as shown. The user 10 then activates the pill delivery mechanism to deliver a prescribed or predetermined pill dose 406a into the delivery tube 408. Accordingly, as discussed hereinabove, the at least two contact areas 412, 414 are connected to a power source such that a circuit and, hence, a current path is completed through the user's body as the user makes contact with each of the two contact areas 412, 414 and a current flow is detected by the detector 20, which is coupled to the user 10.

FIG. 12D illustrates one aspect of the medication delivery apparatus 400 for delivering an individual pill from a reservoir of individually sealed pills. The apparatus 400 illustrated in FIG. 12D comprises a housing 402 having a chamber 404 (reservoir or container) portion for storing a plurality of individually sealed pills 406 in a sealed package 418 (shown in phantom for clarity) and a delivery tube 408 or device for delivering an individual pill dose 406a. The apparatus 400 further comprises an electronically enabled pill valve 420, which when actuated by an actuator mechanism 410 (not shown) as described in connection with FIG. 12C, the electronically enabled pill valve 420 controls the delivery of a single pill dose 406a and also removes the pill 406a from the sealed package 418 prior to delivery of the single dose pill 406a into the delivery tube 408. The sealed package 418 keeps the plurality of individually sealed pills 406 in any desired environment, e.g., dry and sealed environment to keep the pills 406 fresh and possibly eliminate the need for refrigeration, for example. In other aspects, the housing 402, and more particularly the chamber 404, may be sealed such that it opens to the environment briefly and only during the delivery of the single pill dose 406a and otherwise remains sealed.

Also illustrated in FIG. 12D are at least two contact areas 412 and 414 that are electrically coupled to the detector 20 via the user's body. The user 10 holds the apparatus 400 in the user's hand and places the apparatus 400 to the user's mouth, thereby making contact with the first contact area 412 with the user's mouth and the second contact area 414 with the user's hand, thereby making contact with the apparatus 400 in at least two locations, as shown. The user 10 then activates a pill delivery mechanism to deliver a prescribed or predetermined pill dose 406a into the delivery tube 408. Accordingly, as discussed hereinabove, the at least two contact areas 412, 414 are connected to a power source such that a circuit and, hence, a current path is completed through the user's body as the user makes contact with each of the two contact areas 412, 414 and a current flow is detected by the detector 20, which is coupled to the user 10.

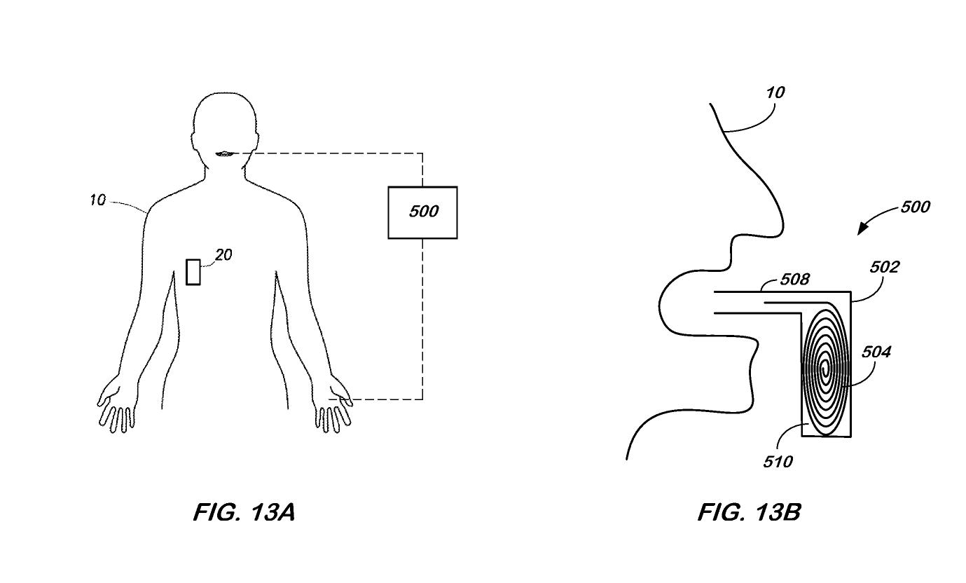

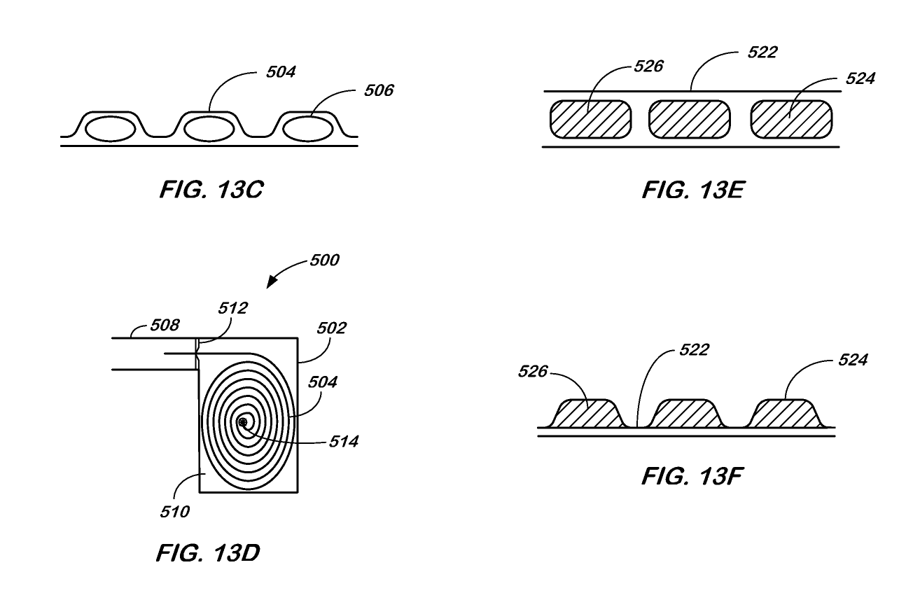

Referring now to FIG. 13A, the user 10 is shown wearing a detector 20 and making physical contact with an apparatus 500. The apparatus 500 is configured for delivery of medication in the form of a pill, tablet, or capsule (pill) contained in a sealed tape roll package. The detector 20 is shown secured at one location on the user's body and is communicatively coupled to the user 10 and is capable of detecting a current flow through the user's body, as discussed hereinabove.

FIG. 13B illustrates one aspect of the medication delivery apparatus 500 for delivering an individual pill from a tape 504 comprising a plurality of pills sealed therein. The apparatus 500 comprises a housing 502 defining a chamber 510 for holding the tape 504 comprising a plurality of pills sealed therein. A pill delivery tube 508 delivers an individual pill dose to the user 10 once the pill has been removed from the tape 504 using a variety of techniques, as discussed hereinbelow, for example. As shown in FIG. 13C, for example, the tape 504 may comprise a plurality of pills 506 sealed within a package that can be coiled up and inserted inside the chamber 510. The apparatus 500 may employ a variety of advancing mechanisms to deliver a single dose or multiple doses in accordance with a prescribed medication plan. The user 10 holds the apparatus 500 in the user's hand and places the apparatus 500 to the user's mouth, thereby making contact with the apparatus 500 in at least two locations. The user 10 then activates a knife and roll delivery mechanism to deliver a prescribed or predetermined pill dose into the delivery tube 508. In the case of a small child, the parent may hold the apparatus 500. In various aspects, if the child makes contact with the parent, then as described hereinbelow, a circuit is completed through the parent and child. The apparatus 500 is shown in block diagram form for clarity of describing the functionality thereof as well as the components therein. However, the apparatus 500 can be made is a variety of shapes according to the various aspects of the present invention.

FIG. 13D illustrates one aspect of the medication delivery apparatus 500 for delivering an individual pill from a tape 504 comprising a plurality of pills sealed therein. The apparatus 500 illustrated in FIG. 13D, shows a knife 512 positioned at a distal portion of the delivery tube 508, relative to the user 10. The knife 512 is configured to strip the pill 506 from the tape 504 roll package as the tape 504 is mechanically advanced for the purpose of delivering a proper pill dose to the user 10. Other mechanical devices may be employed to break open the packaging of the tape 504 and release the pill 506. In one aspect, the roll and knife 512 mechanism may be electronically interlocked to prevent over dosing. In one aspect, an end of tape indicator 514 shows when the medicine is nearing completion and/or when a reorder date is due.

It will appreciated that the tape medication delivery configuration is not limited to holding a pill. As shown in FIGS. 13E and 13F, for example, where FIG. 13E is a top view and

FIG. 13F is a side view of a tape 522 delivery mechanism for liquid medicine. As shown in FIGS. 13E, 13F the tape 522 includes individually sealed pouches 524 containing liquid medication doses 526. Each pocket of the liquid medicine dose 526 is sealed inside the pouches 524 and each pocket full of liquid medicine 526 is individually sealed and attached to a common tape 522 for delivery. A mechanical device, such as the knife 512 (FIG. 13D) breaks open the pouch 526 and delivers the liquid medicine 526 to the user 10 through the delivery tube 508 or other mechanism.

In one aspect, the apparatus 500 as shown in and described in connection with FIGS. 13A, 13C, and 13D may comprise dissolvable medicine doses 506 embedded in a dissolvable tape 504. Accordingly, as the tape 504 is mechanically advanced the knife 512 separates the dissolvable medicine dose 506 and the tape 504 package dissolves.

Although not explicitly shown, for clarity of disclosure it is to be understood that the apparatus 500 as shown in and described in connection with FIGS. 13A and 13D comprises at least two contact areas and that are electrically coupled to the detector 20 via the user's body. As discussed hereinabove, the at least two contact areas could be capacitively coupled to the user. Accordingly, in one aspect, the contact areas may be coated with a thin dielectric, such as plastic, and the electrical communication is accomplished using capacitive coupling through this dielectric to the user. If the contact areas are covered by dielectric, then the apparatus makes contact with the mouth of the user, and the electrodes may or may not be visible to the user. For example, the electrodes could be embedded in the plastic and sense the presence of the mouth using capacitive coupling and looking for a large change in impedance between the two capacitive plates that occurs when the mouth contact both of these plates. Capacitive coupling may be accomplished at signal frequencies greater than about 20 kHz and in some aspects about 80 kHz for good signal-to-noise ratio (SNR, e.g., signal to ambient noise). The user 10 holds the apparatus 500 in the user's hand and places the apparatus 500 to the user's mouth, thereby making contact with a first contact area with the user's mouth and a second contact area with the user's hand, thereby making contact with the apparatus 500 in at least two locations, as shown. The user 10 then activates a roll and knife delivery mechanism to deliver a prescribed or predetermined pill dose into the delivery tube 508. Accordingly, as discussed hereinabove, the at least two contact areas are connected to a power source such that a circuit and, hence, a current path is completed through the user's body as the user makes contact with each of the two contact areas and a current flow is detected by the detector 20, which is coupled to the user 10.

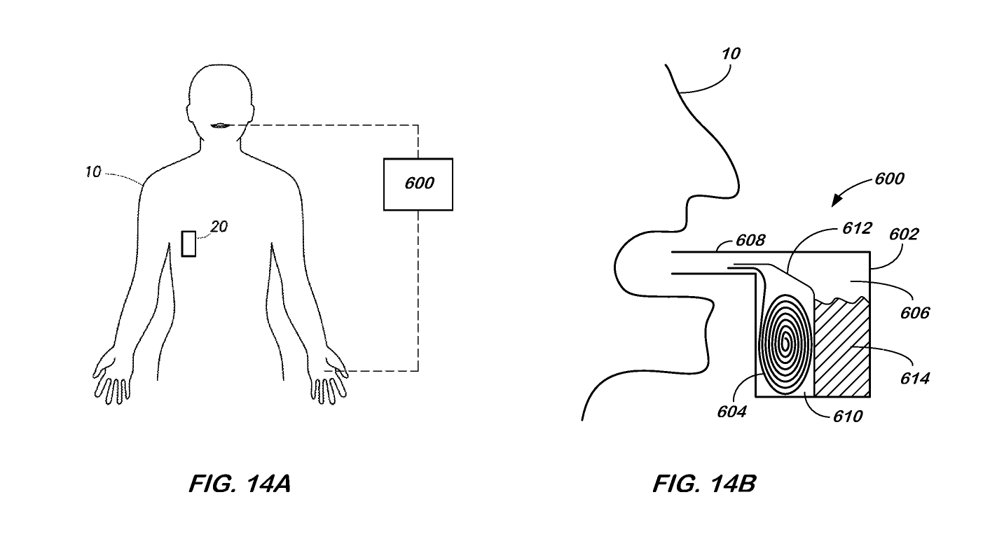

Referring now to FIG. 14A, the user 10 is shown wearing a detector 20 and making physical contact with an apparatus 600. The apparatus 600 is configured for delivery of medication in the form of a combination of liquid and a pill, tablet, or capsule (pill) contained in a sealed tape roll package. The detector 20 is shown secured at one location on the user's body and is communicatively coupled to the user 10 and is capable of detecting a current flow through the user's body, as discussed hereinabove.

FIG. 14B illustrates one aspect of the medication delivery apparatus 600 for delivering an individual pill from a tape 604 comprising a plurality of pills sealed therein while also delivering a dose of liquid 614. The liquid 614 may be water or liquid medication. In one aspect, the liquid 164 may be a two-part liquid medication, where the therapeutic molecules are created or activated by reaction in vivo and/or to control the local pH when the medicine is delivered. The apparatus 600 comprises a housing 602 defining a first chamber 610 for holding the tape 604 comprising a plurality of pills sealed therein and a second chamber 606 divided by a wall 612. The second chamber 606 holds the liquid 614. A delivery tube 608 delivers an individual pill dose in combination with the liquid 614 to the user 10 once the pill has been removed from the tape 604 using a variety of techniques, as discussed hereinbelow, for example. The tape 604 may comprise a plurality of pills sealed within a package that can be coiled up and inserted inside the chamber 610. The apparatus 600 may employ a variety of advancing mechanisms to deliver a single dose or multiple doses in accordance with a prescribed medication plan. In the case of a small child, the parent may hold the apparatus 600. The user 10 holds the apparatus 600 in the user's hand and places the apparatus 600 to the user's mouth, thereby making contact with the apparatus 600 in at least two locations. In various aspects, if the child makes contact with the parent, then as described hereinbelow, a circuit is completed through the parent and child. The apparatus 600 is shown in block diagram form for clarity of describing the functionality thereof as well as the components therein. However, the apparatus 600 can be made is a variety of shapes according to the various aspects of the present invention.

Although not explicitly shown, for clarity of disclosure it is to be understood that the apparatus 600 as shown in and described in connection with FIGS. 14A and 14B comprises at least two contact areas and that are electrically coupled to the detector 20 via the user's body. The user 10 holds the apparatus 600 in the user's hand and places the apparatus 600 to the user's mouth, thereby making contact with a first contact area with the user's mouth and a second contact area with the user's hand, thereby making contact with the apparatus 600 in at least two locations, as shown. The user 10 then activates a roll and knife delivery mechanism to deliver a prescribed pill dose into the delivery tube 608 and then can tilt the housing 602 to release a dose of liquid 614 to wash down the pill dose. Accordingly, as discussed hereinabove, the at least two contact areas are connected to a power source such that a circuit and, hence, a current path is completed through the user's body as the user makes contact with each of the two contact areas and a current flow is detected by the detector 20, which is coupled to the user 10.

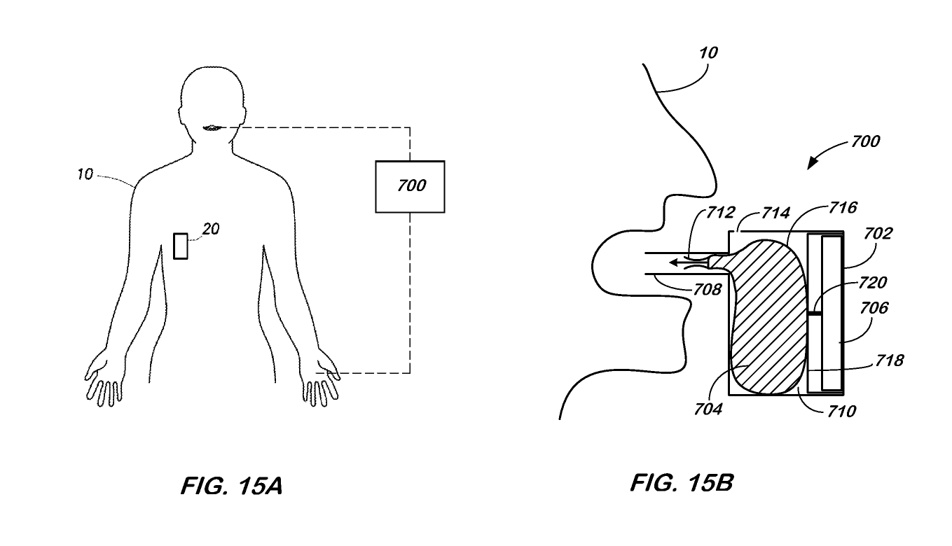

Referring now to FIG. 15A, the user 10 is shown wearing a detector 20 and making physical contact with an apparatus 700. The apparatus 700 is configured for delivery of medication in the form of a combination of liquid and a pill, tablet, or capsule (pill) contained in a sealed tape roll package. The detector 20 is shown secured at one location on the user's body and is communicatively coupled to the user 10 and is capable of detecting a current flow through the user's body, as discussed hereinabove.

FIG. 15B illustrates one aspect of the medication delivery apparatus 700 for delivering an individual does of liquid medication 704 from a bladder 716 (e.g., flexible pouch, bag, etc.). The apparatus 700 comprises a housing 702 defining a chamber 710 for holding the bladder 716 filled with the liquid medication 704. A pressurizing mechanism 706 pressurizes the bladder 716 by a predetermined amount to deliver a corresponding dose of liquid medication 704 to the delivery tube 708 via the flow valve 712. The pressurizing mechanism 706 may comprise hydraulic, pneumatic, mechanical, piston, stepping motor, servo motor, or any other suitable mechanism to apply pressure to the exterior portion of the bladder 716 in order to force a corresponding dose of liquid medicine 704 to flow into the delivery tube 708. In one aspect, as shown, the pressurizing mechanism 706 comprises a movable piston 720 coupled to a movable plate 718. The piston 720 advances the plate 718 to apply pressure to the exterior portion of the bladder 716 and retracts the plate 718 to remove pressure therefrom. The piston 720 may be moved hydraulically, pneumatically, mechanically, electromechanically, electronically, by stepping motors, or by servo motors. The piston 720 may be replaced by a lead screw coupled to a motor (stepping or servo) to advance and retract the plate 718. The pressurizing mechanism 706 may be coupled to a mechanical or electrical actuator. The user 10 holds the apparatus 700 in the user's hand and places the delivery tube 708 of the apparatus 700 to the user's mouth, thereby making contact with the apparatus 700 in at least two locations. The user 10 then activates the actuator to deliver a prescribed or predetermined dose of the liquid medicine 704 into the delivery tube 708. The user 10 then sucks the liquid into his or her mouth. During the sucking operation, air enters through the vent hole 714 and is introduced behind the liquid medicine 704 in the delivery tube 708 to force the liquid medicine 704 into the user's mouth. In various aspects, if the child makes contact with the parent, then as described hereinbelow, a circuit is completed through the parent and child. The apparatus 700 is shown in block diagram form for clarity of describing the functionality thereof as well as the components therein. However, the apparatus 700 can be made is a variety of shapes according to the various aspects of the present invention.

Although not explicitly shown, for clarity of disclosure it is to be understood that the apparatus 700 as shown in and described in connection with FIGS. 15A and 15B comprises at least two contact areas and that are electrically coupled to the detector 20 via the user's body. The user 10 holds the apparatus 700 in the user's hand and places the apparatus 700 to the user's mouth, thereby making contact with a first contact area with the user's mouth and a second contact area with the user's hand, thereby making contact with the apparatus 700 in at least two locations, as shown. Accordingly, as discussed hereinabove, the at least two contact areas are connected to a power source such that a circuit and, hence, a current path is completed through the user's body as the user makes contact with each of the two contact areas and a current flow is detected by the detector 20, which is coupled to the user 10.

The apparatuses 400, 500, 600, 700 as shown in and describe in connection with FIGS. 12A-15B may be include a power source 34, a control unit 36, contact areas 38, 40 and a memory unit 42 as shown in and described in connection with FIGS. 2A and 2B. In accordance with the aspects discussed hereinabove, the housing 402, 502, 602, 702 of the respective apparatuses 400, 500, 600, 700 includes at least two contact areas/points 38, 40 (corresponding to contact areas 412, 414 in FIGS. 12C, 12D) positioned at different locations on the housing. The location and position of the contact points 38, 40 or areas are determined by the shape and design of the apparatus 3 and in accordance with the various aspects of the present invention. Multiple locations for the contact areas 38, 40 are contemplated.

In accordance with another aspect of the present invention, the contact areas 38, 40 (corresponding to contact areas 412, 414 in FIGS. 12C, 12D) may include functionality that allow for reading and recording of biometric information, such as finger print data. This information may be communicated to the apparatuses 400, 500, 600, 700 and stored therein for confirmation of the user's identity.

The contact areas 38, 40 (corresponding to contact areas 412, 414 in FIGS. 12C, 12D) are electrically isolated from each other and at least partially exposed or exposable to allow the user 10 to make contact therewith. In accordance with one aspect of the present invention, the contact areas 38, 40 are positioned such that one contact area 38, for example, makes contact with the user's hand and the other contact area 40, for example, makes contact with the user's mouth. In accordance with alternative aspect of the present invention, additional contact areas may be added to allow for secondary contacts with the hand or mouth as well as to accommodate using a different hand, such as a left hand grip as well or a right hand grip. Furthermore, additional contact areas on the housing 402, 502, 602, 702 can be included to ensure that the apparatus 400, 500, 600, 700 is held properly.

The apparatuses 400, 500, 600, 700 as shown in and described in connection with FIGS. 12A-15B may comprise one or more than one sensor for detecting the delivery of the single dose pill 406a into the mouth of the user 10. For example, sensors may be included that detect liquid, saliva, humidity, changes in coloration as the pill dissolves in the mouth, vacuum, pressure, conductivity, acoustic, among other sensors discussed hereinabove. Such sensors may include additional contact areas for capacitive coupling and detecting user contact with the apparatus 400, electrical field coupling for non-contact detection of the apparatus being brought in proximity to the user 10, among other types of non-contact sensors. In addition, the act of swallowing may be detected by suitably locating the detector 20 or electrodes (e.g., contact areas) coupled to the detector in various locations on the body of the user 10 such s the torso, chest, neck, tongue, palate, among other locations, to detect muscular movements associated with the act of swallowing. In other aspects, an electronic mouth guard outfitted with electrodes may be employed to detect delivery of a pill dose 406a or swallowing the pill dose 406a. Sounds made during the act of swallowing also may be detected with suitable acoustic sensors, as discussed hereinabove, for example. In other aspects, DNA detection techniques, such as optical DNA detection also may be employed to detect the delivery of the single pill dose 406a to the user 10.

In various aspects, the apparatuses 400, 500, 600, 700 as shown in and described in connection with FIGS. 12A-15B may comprise mechanical, electronic, and/or electromechanical to control the delivery of medication only at prescribed scheduled times and only in the prescribed amounts to prevent overdosing. For example, a lock-out mechanism may be incorporated into the medication delivery apparatuses 400, 500, 600, 700 without the ability for the user 10 to break into the housing to access the medication outside of the prescribed window of time after a proper dose had been delivered. For example, a pill to be dispensed only in the morning could not be dispensed in the afternoon and the user cannot break-into the housing or otherwise access the chamber, pill repository, or reservoir to access the medication.

In aspects when an IEM is delivered by the medicinal delivery apparatuses 400, 500, 600, 700 as shown in and described in connection with FIGS. 12A-15B, the IEM generates its own power and communicates with the detector 20 (FIGS. 1A, 3A, 3E, 5A, 10, 12A, 13A, 14A, 14A, 15A, for example) and by altering conductance resulting in an alteration of the characteristics of the current flow. The altered characteristics contain the information encoded in the current flow, as disclosed in U.S. patent application Ser. No. 12/564,017 entitled COMMUNICATION SYSTEM WITH PARTIAL POWER SOURCE filed on Sep. 21, 2009, and published as US 2010/0081894, the entire specification of which is incorporated herein by reference. The encoded information is then detected by the detector 20 and decoded. In addition, information disclosed in U.S. patent application Ser. No. 11/912,475 entitled PHARMA-INFORMATICS SYSTEM filed on Apr. 28, 2006, and published as US 2008/0284599 the entire specification of which is incorporated herein by reference.

Accordingly, in one aspect, the medicinal delivery apparatuses 400, 500, 600, 700 as shown in and described in connection with FIGS. 12A-15B are configured to deliver an IEM system for indicating the occurrence of an event, such as an indication that a medicinal dose has been ingested by the user 10. In one aspect, the IEM system is used with a conducting fluid to indicate the event marked by contact between the conducting fluid and the IEM system. For example, the IEM system may be used with pharmaceutical product and the event that is indicated is when the product is taken or ingested and the IEM the system is introduced to an environment that contains a conducting fluid.

When the product that includes the IEM system is taken or ingested, the device comes into contact with the conducting liquid of the body. When the IEM system comes into contact with the body fluid, a voltage potential is created and the system is activated. A portion of the power source is provided by the device, while another portion of the power source is provided by the conducting fluid.

An ingestible product that includes an IEM system may be configured as an orally ingestible pharmaceutical formulation in the form of a pill or capsule. Upon ingestion, the pill moves to the stomach. Upon reaching the stomach, the product is in contact with stomach fluid and undergoes a chemical reaction with the various materials in the stomach fluid, such as hydrochloric acid and other digestive agents. Although the IEM system is discussed in reference to a pharmaceutical environment, the scope of the disclosed aspects of the IEM system is not limited thereby. The IEM system can be used in any environment where a conducting fluid is present or becomes present through mixing of two or more components that result in a conducting liquid.

A pharmaceutical product may be combined with an IEM system or an ionic emission module. The IEM system uses the voltage potential difference to power up and thereafter modulates conductance to create a unique and identifiable current signature. Upon activation, the IEM system controls the conductance and, hence, current flow to produce the current signature. The encoded information is then detected by the detector 20 and decoded.

In one specific example, the IEM system combined with a pharmaceutical product, as the product or pill is ingested, the IEM system is activated. The IEM system controls conductance to produce a unique current signature that is detected, thereby signifying that the pharmaceutical product has been taken. The IEM system includes a framework, that acts as a chassis for the IEM system and multiple components are attached to, deposited upon, or secured to the framework. In this aspect, a digestible material is physically associated with the framework. The material may be chemically deposited on, evaporated onto, secured to, or built-up on the framework all of which may be referred to herein as “deposit” with respect to the framework. The material is deposited on one side of the framework. The materials of interest that can be used as material include, but are not limited to: Cu or Cul. The material is deposited by physical vapor deposition, electrodeposition, or plasma deposition, among other protocols. The material may be from about 0.05 to about 500 μm thick, such as from about 5 to about 100 μm thick. The shape is controlled by shadow mask deposition, or photolithography and etching. Additionally, even though only one region is shown for depositing the material, each system may contain two or more electrically unique regions where the material may be deposited, as desired.

At a different side, which is the opposite side, for example, another digestible material is deposited, such that the two ingestible materials are dissimilar. The different side selected may be the side next to the side selected for the material. The scope of the present invention is not limited by the side selected and the term “different side” can mean any of the multiple sides that are different from the first selected side. The dissimilar materials may be selected such that they produce a voltage potential difference when the IEM system is in contact with conducting liquid, such as body fluids. The materials of interest for the first material include, but are not limited to: Mg, Zn, or other electronegative metals. As indicated above with respect to the first material, the second material may be chemically deposited on, evaporated onto, secured to, or built-up on the framework. Also, an adhesion layer may be necessary to help the material(s) to adhere to the framework. Typical adhesion layers for the material(s) are Ti, TiW, Cr or similar material. Anode material and the adhesion layer may be deposited by physical vapor deposition, electrodeposition or plasma deposition. The second (anode) material may be from about 0.05 to about 500 μm thick, such as from about 5 to about 100 μm thick. However, the thickness or deposition process are not limiting aspects of the IEM system or any of the materials used to deposit or secure the materials to the framework.

According to one aspect, the dissimilar materials can be any pair of materials with different electrochemical potentials. Additionally, wherein the IEM system is used in-vivo, the dissimilar materials may be vitamins that can be absorbed. More specifically, the dissimilar materials can be made of any two materials appropriate for the environment in which the IEM system will be operating. For example, when used with an ingestible product, the dissimilar materials are any pair of materials with different electrochemical potentials that are ingestible. An illustrative example includes the instance when the IEM system is in contact with an ionic solution, such as stomach acids. Suitable materials are not restricted to metals, and in certain embodiments the paired materials are chosen from metals and non-metals, e.g., a pair made up of a metal (such as Mg) and a salt (such as CuCI or Cul). With respect to the active electrode materials, any pairing of substances—metals, salts, or intercalation compounds—with suitably different electrochemical potentials (voltage) and low interfacial resistance are suitable.

In various aspects, the apparatus 3, 25, 30, 400, 500, 600, 700 as shown in and described in connection with respective FIGS. 1A, 3A, 5A, 12A-15B may comprise a transconduction unit is provided in communication with a control unit 36 (controller, processing unit, processor, state machine) as shown in and described in connection with FIGS. 2A, 2B, 4A, 4B, and 6A, 6B. The transconduction unit comprises at least two contact areas electrically isolated from each other and positioned to allow contact with each of the contact areas, and a power source. The at least one contact area is electrically coupled to one terminal of the power source and at least one other contact area is electrically coupled to another terminal of the power source. A current path is completed through the user's body to allow current flow as the user makes contact with each of the two contact areas and current flow is detected by the controller.

In one aspect, the transconduction unit further comprises a control module electrically connected between the power source and one of the at least two contact areas. The control module is configured to encode information in the current flow.

In one aspect, the apparatus 3, 25, 30, 400, 500, 600, 700 (as described in connection with FIGS. 1A-7C and FIGS. 12A-15B hereinabove) may be configured to confirm delivery of a medicinal dose to a user. The apparatus comprises at least two contact areas that are electrically isolated from each other, a power source including a first terminal and second terminal, wherein at least one contact area is electrically coupled to the first terminal and at least one other contact area is electrically coupled to the second terminal, and a control module electrically connected between the first terminal of the power source and one of the contact areas. The control module is configured to vary the conductance of a current path that is created once the user makes contact with each of the two contact areas. A current path is completed through the user's body as the user makes contact with each of the two contact areas. The variation in the conductance encodes information in the current flow.

The detectors of interest include, but are not limited to, those receivers disclosed in: PCT application serial no. PCT/US2006/016370 published as WO 2006/116718; PCT application serial No. PCT/2007/24225 published as WO 2008/063626; PCT application serial no. PCT/US2008/52845 published as WO/2008/095183; the disclosures of which applications are herein incorporated by reference.

In accordance with other aspects of the present invention, the system may include two or more (such as three or more, including four or more) detectors. In such systems, the two or more detectors may be adaptively arranged at any desired location on the body of the user. For example, all of the body-associated detectors may be present on the same side of a body, such as the front torso of a body, or they may be present on opposite sides of a body, such as the front and back of the torso of a body. In the specific examples where the detector 20 is receiving acoustic information from within the lungs or the acoustic unit 33 of FIG. 4C or the acoustic unit 31 of FIG. 5E, which information is associated with the user inhaling, then the detectors are positioned about the torso, proximal to the lungs. In other aspects, the detector 20 associated with the apparatuses 400, 500, 600, 700 may be located in a position on the user's body to detect the swallowing action of the user 10.

In accordance with another aspect of the present invention, where the detector 20 includes the accelerometer 90 that monitors the motion of the lungs associated with a rapid inhale, then the detectors are positioned close to the lungs. If the detector 20 has also detected the presence of a current indicative of the user holding the apparatus 3, 25, 30, 400, 500, 600, 700 (as described in connection with FIGS. 1A-7C and FIGS. 12A-15B hereinabove), then the detector 20 records the occurrence of the event associated with the motion of the lungs. In such an example, the detector 20 would be positioned on the user's body in a location that allows best detection of the lung's motion. The lung motion may be employed to determine whether the user 10 has swallowed the prescribed dose delivered by the medicine delivery devices discussed hereinabove.

Depending on the needs of a particular application, the current detected by the detector 20 may be generic, such that it merely identifies that the apparatus 3, 25, 30, 400, 500, 600, 700 (as described in connection with FIGS. 1A-7C and FIGS. 12A-15B hereinabove) has contacted the target sites, which is the user's mouth and limb. In these instances, each apparatus 3, 25, 30, 400, 500, 600, 700 can encode unique information in the current flow that uniquely identifies that particular device relative to all the other devices, especially if the user is using multiple devices.

The apparatus 3, 25, 30, 400, 500, 600, 700 (as described in connection with FIGS. 1A-7C and FIGS. 12A-15B hereinabove) is configured to generate a variety of different types of signals, including but not limited to: current signatures produced through controlling conductance, RF signals, magnetic signals, conductive (near field) signals, acoustic signals, etc. The transmission time may vary, where in certain instances the transmission time may range from 0.1 μsec to 48 hours or longer, including from 1 minute to 10 minutes. Depending on the given aspect, the identifier may produce a unique current signature once. Alternatively, the identifier may be configured to produce a unique current signature with the same information (identical signals), two or more times, where the collection of discrete identical signals may be collectively referred to as a redundant signal.

In certain aspects, the components or functional blocks of the detector and the apparatus are present on integrated circuits, where the integrated circuits include a number of distinct functional blocks, i.e., modules and at least some of, e.g., two or more, up to an including all of, the functional blocks may be present in a single integrated circuit. By single integrated circuit is meant a single circuit structure that includes all of the different functional blocks. As such, the integrated circuit is a monolithic integrated circuit that is a miniaturized electronic circuit (which may include semiconductor devices, as well as passive components) that has been manufactured in the surface of a thin substrate of semiconductor material. The integrated circuits of certain aspects of the present invention may be hybrid integrated circuits, which are miniaturized electronic circuits constructed of individual semiconductor devices, as well as passive components, bonded to a substrate or circuit board.

Although the foregoing invention has been described in some detail by way of illustration and example for purposes of clarity of understanding, the following description is directed to additional aspects of the present invention in a more general form. Accordingly, in one aspect, a system is provided to deliver a medicinal dose to a user and to confirm delivery of the dose. The system comprises a detector configured to couple to the user and an apparatus. The apparatus includes a housing defining a chamber to store the dose, at least two contact areas positioned on the housing and the at least two contact areas are electrically isolated from each other, and a power source secured to the housing and including a positive phase terminal and a negative phase terminal, wherein at least one contact area is electrically coupled to the positive terminal and at least one other contact area is electrically coupled to the negative terminal. In one aspect, the contact areas may be at least partially exposed on the exterior of the housing. A current path is completed through the user's body as the user makes contact with each of the two contact areas and current flow is detected by the detector that is coupled to the user.

In one aspect, the apparatus further comprises a control module electrically connected between the power source and one of the at least two contact areas, wherein the control module is configured to control information output from the apparatus.

In one aspect, the control module is configured to vary the conductance of the current path to encode information in the current flow and the detector decodes the information.

In one aspect, the apparatus further comprises a memory unit electrically connected to the control module to store information associated with delivery of the dose to the user.

In one aspect, the apparatus further comprises a transceiver electrically coupled to the control module wherein information can be transmitted and/or received from the apparatus to the detector other than through the current flow.

In one aspect, the detector is configured to be implanted within the user's body. The detector comprises a hermetically sealed housing, a power source secured within the housing, a processor electrically coupled to the power source and secured within the housing, and at least one sensing probe secured to the housing wherein the probe is at least partially exposed to contact the user's tissue. The probe is electrically coupled to the processor so that the processor detects physiological parameters associated with the user and the current flow through the user. A memory unit is electrically coupled to the processor and secured within the housing to store data. The transceiver is electrically coupled to the processor and secured within the housing to receive and decode information transmitted from the apparatus.

In one aspect, the detector is configured to be secured to the user's skin. The detector comprises a housing, a power source secured to the housing, a processor electrically coupled to the power source and secured to the housing, at least one sensing probe secured to the housing wherein the probe is at least partially exposed to contact the user's skin, wherein the probe is electrically coupled to the processor to allow the processor to detect physiological parameters associated with the user and the current flow through the user, and a memory unit is electrically coupled to the processor and secured to the housing to store data. The transceiver is electrically coupled to the processor and secured to the housing to receive and decode information transmitted from the apparatus.

In one aspect, the transceiver provides an activation signal to the detector and the detector includes an accelerometer electrically coupled to the processor and secured to the housing. The accelerometer is activated by the processor to detect motion upon the detector receiving the activation signal from the apparatus.

In one aspect, the transceiver includes an optical communication apparatus that encodes information in an optical beam and wherein the detector includes an optical receiver electrically coupled to the processor and secured to the housing. The optical receiver captures the optical beam and decodes the information.

In one aspect, the transceiver provides wireless communication of the data from the detector to a data management center.

In one aspect, the transceiver provides an activation signal to the detector and the detector further comprises an acoustic module electrically connected to the processor and secured to the housing. The acoustic module is activated by the processor to detect acoustic information from the use's lungs upon the detector receiving the activation signal from the apparatus.

In one aspect, an apparatus is provided to deliver a medicinal dose to a user and track the timing of the delivery of the dose. The apparatus comprises a housing that defines a chamber to store the dose, at least two contact areas positioned on the exterior of the housing wherein the contact areas are configured to make contact with the user and wherein the contact areas are electrically insulated from each other, and a power source secured to the housing to provide power to produce a current flow through the user's body, wherein the power source includes two terminals and each terminal is electrically coupled to one contact area. The circuit is completed as the user's skin contacts each of the at least two contact areas to create a circuit causing a current flow through the user's body.

In one aspect, the apparatus further comprises a control module secured to the housing and electrically coupled to each of the two contact areas. The control module alters the conductance of the current path between the two terminals to vary the current flow characteristics through the user thereby encoding information in the current flow.

In one aspect, the apparatus further comprises a memory unit electrically coupled to the control module and secured to the housing. The memory module stores information associated with completing the circuit through the user's body and delivery of the dose.

In one aspect, the apparatus further comprises a transceiver electrically coupled to the control module and secured to the housing. The transceiver unit allows the apparatus to transmit and/or receive information associated with the delivery of the dose to the user.

In one aspect, the transceiver includes an optical transmitter module for optical communication.

In one aspect, the transceiver includes a wireless transmitter module for wireless communication.

In one aspect, the transceiver encodes information from the memory unit and transmits that information to a system external to the apparatus.

In one aspect, the transceiver sends an activation signal to the external system once a circuit is completed through the user's body wherein the activation signal is an indicator that the user is holding the apparatus and prepared to initiate delivery of the dose.

In one aspect a method is provided for recording the time that a medicinal dose is taken by a user. The method comprises the steps of activating a power module of a apparatus when the user makes contact with the exterior of the apparatus in such a manner to complete a circuit to allow for current flow between two terminals of the power module and through the user's body, altering the current characteristics through changes in the conductance of the circuit that is formed through the user's body using a conductance control module, detecting the current characteristics through the user's body using a detector, the current characteristics includes information associated with at least one of the apparatus and the dose, and recording the timing of delivery of the inhalable dose.

In one aspect, an apparatus is provided to deliver a medicinal dose to a user and to confirm delivery of the dose. The apparatus comprises a housing defining a chamber to store the dose, a power source secured to the housing and including a positive terminal and a negative terminal, a control module electrically coupled to the power source, an acoustic detector secured to the housing and electrically coupled to the control module, wherein the acoustic detector detects vibrations, and at least two contact areas positioned on the housing wherein the contact areas are at least partially exposed on the exterior of the housing and the at least two contact areas are electrically isolated from each other. The at least one contact area is electrically coupled to one terminal of the power source and at least one other contact area is electrically coupled to the other terminal. A current path is completed through the user's body as the user makes contact with each of the two contact areas and current flow is detected by the control module.

In one aspect, the control module is electrically connected between the power source and one of the at least two contact areas. The control module is configured to control conductance to encode information in the current flow.

In one aspect, the control module is electrically coupled to the at least two contact areas and to both terminals of the power source.

In one aspect, the acoustic detector is secured to the outside of the housing.

In one aspect, the apparatus further comprises a memory unit electrically connected to the control module to store information associated with delivery of the dose to the user.

In one aspect, the apparatus further comprises a transceiver electrically coupled to the control module wherein information can be transmitted and/or received from the apparatus to an external computer other than through the current flow.

In one aspect, the acoustic detector is secured inside the housing.

In one aspect, the acoustic detector provides an activation signal to the control module upon detection of a vibration representing the loading of the medication into the chamber.

In one aspect, the acoustic detector provides an activation signal to the control module and the control module records acoustic information associated with the user inhaling. The information is provided to the control module through the acoustic detector.

In one aspect, the control module provides a unique time stamp associated with the delivery of the dose and wherein the control module receives an identifier signal that is associated with the user wherein the combination of the time stamp and the identifier signal confirms deliver of the dose to the user.

In one aspect, an apparatus is provided to deliver a dose to a user and track the timing of the delivery of the dose. The apparatus comprises a housing defining a chamber to store the dose, at least two contact areas positioned on the housing wherein the contact areas are at least partially exposed on the exterior of the housing and the at least two contact areas are electrically isolated from each other, a power source secured within the housing to provide power to produce a current flow through the user's body, wherein the power source includes two terminals and wherein each terminal is electrically coupled to one contact area, a control module electrically coupled to the power source, wherein the control module enters a sleep mode while the apparatus is inactive, and an acoustic detector secured to the housing and electrically coupled to the control module, wherein the acoustic detector detects vibrations and sends an activation signal to the control module to activate the control module. A sound of the dose being loaded into the chamber activates the control module and wherein a current path is completed through the user's body as the user makes contact with each of the contact areas indicating that the user is about the receive a dose of the medication.

In one aspect, the apparatus further comprises a memory unit electrically coupled to the control module and secured to the housing. The memory module stores information associated with delivery of the dose to the user.

In one aspect, the apparatus further comprises a transmitter unit electrically coupled to the control module and secured to the housing. The transmitter unit allows the apparatus to transmit information.

In one aspect, the apparatus further comprises an optical detector module for detecting that a dose was delivered to the user and the optical information is compared to acoustic information associated with the sound vibrations generated from inhaling through the apparatus to confirm delivery of the dose to the user.

In one aspect, a system is provided to deliver a medicinal dose to a user and to confirm delivery of the dose. The system comprises a detector including a capacitive coupler, wherein the detector is worn by the user, and an apparatus comprising a housing defining a chamber to store the dose, a power source secured to the housing and including a positive terminal and a negative terminal, wherein the power source includes an isolating source that produces a carrier wave, a control module electrically coupled to the power source, wherein the control module alters the characteristics of the isolating source to encode information in the carrier wave, and at least two areas positioned on the housing wherein one area is a partially exposed contact area and one area is capacitive coupled area, wherein the two areas are electrically isolated from each other. The one output of the isolating source is coupled to the contact area and the other output of the isolating source is coupled to the capacitive coupled area. The contact area is touched by the user and the capacitive coupled area is capacitively coupled to the capacitive coupler worn by the user. A portion of the carrier wave's path is through the user's body using the contact area and a portion of carrier wave's path is through capacitive conductance using the capacitive coupling between the capacitive coupled area and the capacitive coupler worn by the user. The same principle described here for the detector also may be applied to an actuator located on the medicine delivery device apparatus.

In one aspect, the housing defines an aperture to generate an acoustic wave as the user inhales through the apparatus and wherein the detector further comprises an acoustic detector for detecting acoustic wave associated with the user inhaling through the apparatus, which acoustic waves traveling through the user's body and through the air, wherein the acoustic detector correlates the acoustic wave through the user's body with the acoustic wave through the air to confirm delivery of the dose to the user.

In one aspect, a system is provided to deliver a medicinal dose to a user and to confirm delivery of the dose. The system comprises a detector coupled to the user, wherein the detector gathers physiological information about the user to create a personal profile about the user, an apparatus, wherein the apparatus includes a housing defining a chamber to store the dose and an opening through which the user inhales to receive the dose, and an acoustic unit for producing an acoustic signal. The acoustic unit is secured to the housing of the apparatus. The acoustic unit comprise a support layer comprising an adhesive layer on one surface for securing the acoustic unit to the apparatus, a vibration detection unit secured to the support layer to detect acoustic information produced by the apparatus and the user inhaling through the apparatus and produce a detection signal, a controller secured to the support layer and in communication with the vibration detection unit, wherein the controller receives the detected signal from the vibration detection unit and produces a digital signal representing the detected signal, a sound generation unit secured to the support layer and in communication with the controller, wherein the sound generation unit receives the digital signal and produces the acoustic signal, and a top layer secured to the support layer to define a cavity that contains and protects the vibration unit, the controller, and the sound generation unit within the cavity. The acoustic signal represents information associated with the inhalable dose being loaded into the chamber and the user inhaling through the apparatus and wherein the acoustic signal is detected by the detector to confirm delivery of the dose.

In one aspect, the system further comprises at least two contact areas positioned on the housing. The contact areas are at least partially exposed on the exterior of the housing and the at least two contact areas are electrically isolated from each other. The acoustic unit includes a power source. The at least one contact area is electrically coupled to a positive terminal of the power source and at least one other contact area is electrically coupled to the negative terminal. A current path is completed through the user's body as the user makes contact with each of the two contact areas and current flow is detected by the detector that is coupled to the user.

In one aspect, the controller is configured to control information output from the acoustic unit.

In one aspect, the controller is configured to vary the conductance of the current path to encode information in the current flow.

In one aspect, the acoustic unit further comprises a memory unit electrically connected to the controller to store.

In one aspect, the detector is configured to be implanted within the user's body. The detector comprises a hermetically sealed housing, a power source secured within the housing, a processor electrically coupled to the power source and secured within the housing, at least one sensing probe secured to the housing wherein the probe is at least partially exposed to contact the user's tissue, wherein the probe is electrically coupled to the processor so that the processor detects physiological parameters associated with the user and the current flow through the user, a transceiver module electrically coupled to the processor and secured within the housing to receive and decode information transmitted from the apparatus, and a memory unit electrically coupled to the processor and secured within the housing to store data.

In one aspect, the detector is configured to be secured to the user's skin. The detector comprises a housing, a power source secured to the housing, a processor electrically coupled to the power source and secured to the housing, at least one sensing probe secured to the housing, wherein the probe is electrically coupled to the processor to allow the processor to detect physiological parameters associated with the user and the current flow through the user, a transceiver module electrically coupled to the processor and secured to the housing to receive and decode information transmitted from the apparatus, and a memory unit electrically coupled to the processor and secured to the housing to store data.

In one aspect, the system further comprises an apparatus secured to the housing of the apparatus. The apparatus provides an activation signal to the detector and the detector includes an accelerometer electrically coupled to the processor and secured to the housing of the detector. The accelerometer is activated by the processor of the detector to detect motion upon the detector receiving the activation signal from the apparatus.

In one aspect, the transceiver module provides wireless communication of the data from the detector to a data management center.

In one aspect, an apparatus is provided for detection of delivery of inhalable medication to a user. The apparatus comprises a vibration detection unit to detect acoustic information and produce a detection signal, a controller in communication with the vibration detection unit, wherein the controller receives the detection signal from the vibration detection unit and produces a digital signal representing the detection signal, a sound generation unit secured to the support layer and in communication with the controller, wherein the sound generation unit receives the digital signal and produces an acoustic signal that indicates delivery of the inhalable dose.

In one aspect, the apparatus further comprises a transmission unit in communication with the controller. The transmission unit receives the digital signal and communicates with a wireless apparatus to indicate the inhalation event has occurred.

In one aspect, the apparatus further comprises a transconduction unit in communication with the controller. The transconduction unit comprises at least two contact areas electrically isolated from each other and positioned to allow contact with each of the contact areas, and a power source. The at least one contact area is electrically coupled to one terminal of the power source and at least one other contact area is electrically coupled to another terminal of the power source. A current path is completed through the user's body to allow current flow as the user makes contact with each of the two contact areas and current flow is detected by the controller.

In one aspect, the transconduction unit further comprises a control module electrically connected between the power source and one of the at least two contact areas. The control module is configured to encode information in the current flow.

In one aspect, an apparatus is provided to confirm delivery of a medicinal dose to a user. The apparatus comprises at least two contact areas that are electrically isolated from each other, a power source including a first terminal and second terminal, wherein at least one contact area is electrically coupled to the first terminal and at least one other contact area is electrically coupled to the second terminal, and a control module electrically connected between the first terminal of the power source and one of the contact areas. The control module is configured to vary the conductance of a current path that is created once the user makes contact with each of the two contact areas. A current path is completed through the user's body as the user makes contact with each of the two contact areas. The variation in the conductance encodes information in the current flow.

In one aspect a method is provided for recording the time that a medicinal dose is taken by a user. The method comprises the steps of activating a power module of an apparatus when the user makes contact with the exterior of the apparatus in such a manner to complete a circuit to allow for current flow between two terminals of the power module and through the user's body, altering the current characteristics through changes in the conductance of the circuit that is formed through the user's body using a conductance control module, generating an acoustic signal using an acoustic unit, detecting the acoustic signal using a detector, wherein the acoustic signal includes information associated with delivery of the dose to the user, and recording the timing of delivery of the inhalable dose.

Depending on the particular application, the detector may be positioned in a variety of different configurations relative to the organ of interest. For example, where a single body-associated signal detector is employed, the methods may include initially positioning or implanting the single receiver at a location proximal to the organ of interest. Where the organ of interest is the lung, the single receiver may be positioned near the lungs, as desired. With other systems that include two or more signal detectors, the detectors may be positioned at a variety of body locations. For example, the methods may include positioning two or more distinct detectors at distinct locations near the lungs or positioning one detector at a front abdominal location and a second detector at a back location. This latter configuration is representative of instances where the detectors are placed on opposite sides of a target organ, e.g., to measure impedance through the organ, motion, or sound.

It is to be understood that this invention is not limited to particular aspects described, and as such may vary. It is also to be understood that the terminology used herein is for the purpose of describing particular aspects only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Certain ranges have been presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

As will be apparent to those of skill in the art upon reading this disclosure, each of the individual aspects described and illustrated herein has discrete components and features which may be readily separated from or combined with the features of any of the other several aspects without departing from the scope of the present invention. Any recited method can be carried out in the order of events recited or in any other order which is logically possible.

Although the foregoing aspects of the present invention have been described in some detail by way of illustration and example for purposes of clarity of understanding, it is readily apparent to those of ordinary skill in the art in light of the teachings of the various aspects of the present invention that certain changes and modifications may be made thereto without departing from the scope of the appended claims.

1-36. (canceled)

37. An apparatus comprising:
a housing that defines a chamber;
a first contact area and a second contact area coupled to the chamber wherein the first and second contact areas are electrically insulated from each other; and
a power source secured to the housing and electrically coupled to the first and second contact areas,
wherein a circuit is completed through a user's body to produce a current flow when the user contacts the first and the second contact areas.

38. The apparatus of claim 37, further comprising a control module secured to the housing and electrically coupled to the first and second contact areas, wherein the control module alters a conductance of the current flow to encode information in the current flow.

39. The apparatus of claim 38, wherein the control module provides a unique time stamp associated with the delivery of a dose and receives an identifier signal that is associated with a user wherein the combination of the time stamp and the identifier signal confirms delivery of the dose to the user.

40. The apparatus of claim 38, further comprising a memory unit electrically coupled to the control module and secured to the housing, wherein the memory module stores information associated with the dose.

41. The apparatus of claim 38, further comprising a transceiver electrically coupled to the control module and secured to the housing, wherein the transceiver allows the apparatus to transmit and/or receive information associated with the delivery of the dose to the user.

42. The apparatus of claim 41, wherein the transceiver comprises at least one of an optical transmitter module for optical communication or a wireless transmitter module for wireless communication.

43. The apparatus of claim 41, wherein the transceiver encodes information from the memory unit and transmits that information to a device external to the apparatus.

44. The apparatus of claim 43, wherein the transceiver sends an activation signal to the device once the circuit is completed through the user's body wherein the activation signal is an indicator that the user is prepared to initiate delivery of the dose.

45. The apparatus of claim 37, further comprising an acoustic unit secured to the housing.

46. The apparatus of claim 45, wherein the acoustic unit is activated by a processor to detect acoustic information from the user's lungs upon the acoustic unit receiving the activation signal from the apparatus.

47. The apparatus of claim 45, wherein the acoustic unit comprises:
a support layer comprising an adhesive layer on one surface for securing the acoustic unit to the apparatus;
a vibration detection unit secured to the support layer to detect acoustic information produced by the apparatus and the user inhaling through the apparatus and produce a detection signal;
a controller secured to the support layer and in communication with the vibration detection unit, wherein the controller receives the detected signal from the vibration detection unit and produces a digital signal representing the detected signal;
a sound generation unit secured to the support layer and in communication with the controller, wherein the sound generation unit receives the digital signal and produces the acoustic signal; and
a top layer secured to the support layer to define a cavity that contains and protects the vibration unit, the controller, and the sound generation unit within the cavity,
wherein the acoustic signal represents information associated with the inhalable dose being loaded into the chamber and the user inhaling through the apparatus and wherein the acoustic signal is detected by the acoustic unit to confirm delivery of the dose.

48. The apparatus of claim 39, wherein the dose is an inhalable dose.

49. The apparatus of claim 39, wherein the dose is an ingestible dose.

50. The apparatus of claim 37, further comprising:
a detector configured to be associated with a user and configured to detect the current flow through the user's body.

51. The apparatus of claim 47, wherein the acoustic unit is coupled to a control module.

52. The apparatus of claim 51, wherein the acoustic unit provides an activation signal to the control module upon detection of a vibration representing the loading of a medication into the chamber.

53. The apparatus of claim 51, wherein a sound of the dose being loaded into the chamber activates the control module and wherein a current path is completed through the user's body as the user makes contact with each of the contact areas indicating that the user is about the receive a dose of a medication.

54. The apparatus of claim 51, wherein the acoustic unit provides an activation signal to the control module and the control module records acoustic information associated with the user inhaling, wherein the information is provided to the control module through the acoustic detector.

55. The apparatus of claim 37, further comprising:
an isolating source that produces a carrier wave;
a control module configured to alter the characteristics of the isolating source to encode information in the carrier wave; and
wherein the second contact area is a capacitive coupled area;
wherein a first output of the isolating source is coupled to the first contact area and a second output of the isolating source is coupled to the second, capacitive coupled contact area,
wherein the first contact area is touched by the user and the capacitive coupled area is capacitively coupled to a capacitive coupler worn by the user, and
wherein a portion of the carrier wave's path is through the user's body using the first contact area and a portion of carrier wave's path is through capacitive conductance using the capacitive coupling between the second, capacitive coupled contact area and the capacitive coupler worn by the user.

56. The apparatus of claim 54, wherein the detector comprises a vibration detection unit to detect acoustic information and produce a detection signal;
a controller in communication with the vibration detection unit, wherein the controller receives the detection signal from the vibration detection unit and produces a digital signal representing the detection signal;
a sound generation unit secured to the support layer and in communication with the controller, wherein the sound generation unit receives the digital signal and produces an acoustic signal that indicates delivery of the dose.
